(19) United States
(12) Patent Application Publication
(10) Pub. No.: US 2023/0015226 A1
(43) Pub. Date: Jan. 19, 2023

(54) PRIMER EXCHANGE REACTION IN A MATRIX-EMBEDDED SAMPLE

(71) Applicant: 10x Genomics, Inc., Pleasanton, CA (US)

(72) Inventors: Hong CHEN, Dublin, CA (US); Evan R. DAUGHARTHY, Cambridge, MA (US); Zhao ZHAO, Newton, MA (US)

(21) Appl. No.: 17/852,250

(22) Filed: Jun. 28, 2022

Related U.S. Application Data
(60) Provisional application No. 63/216,445, filed on Jun. 29, 2021.

(51) Int. Cl. C12Q 1/6841

(52) U.S. Cl. CPC C12Q 1/6841; C12Q 1/682

(57) ABSTRACT

The present application provides methods, compositions, and kits for analyzing a biological sample embedded in a three-dimensional polymerized matrix using a primer-exchange reaction (PER). In some embodiments, the methods comprise contacting the sample with a nucleic acid molecule that directly or indirectly binds to an analyte in the sample and immobilizing the nucleic acid molecule in the matrix, wherein the nucleic acid molecule comprises a free 3′ priming region for initiation of PER. In some embodiments, the methods enable sensitive detection of the identity and relative position of analytes in the sample.

### CROSS-REFERENCE TO RELATED APPLICATIONS

This application claims priority to U.S. Provisional Patent Application No. 63/216,445, filed Jun. 29, 2021, entitled “PRIMER EXCHANGE REACTION IN A MATRIX-EMBEDDED SAMPLE,” which is herein incorporated by reference in its entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 202412006600SEQLIST.TXT, date recorded: Jun. 28, 2022, size: 2,106 bytes).

### FIELD

The present disclosure relates in some aspects to methods for analyzing a sample embedded in a three-dimensional polymerized matrix using a primer-exchange reaction (PER).

### BACKGROUND

Analysis of the cellular and subcellular distribution of gene products such as RNA and proteins in situ in a biological sample provides important clues as to their function. In situ hybridization-based approaches are limited by technical considerations such as probe penetration and signal detection in tissue samples. There is a need for new and improved methods for in situ assays that preserve spatial localization of analytes and allow for signal amplification, enabling detection and reconstruction of the identity and three-dimensional positional information of analytes in the sample. The present disclosure addresses these and other needs.

### BRIEF SUMMARY

In some aspects, provided herein is a method for analyzing a sample embedded in a three-dimensional polymerized matrix, comprising: a) contacting the sample with a nucleic acid molecule, wherein the nucleic acid molecule comprises a free 3′ priming region; b) immobilizing the nucleic acid molecule to the matrix; c) contacting the sample with: (i) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the nucleic acid molecule is complementary to the 3′ toehold domain of the initial hairpin molecule, and (ii) a polymerase having strand displacement activity; d) incubating the sample under conditions for polymerization to produce an elongated product of the immobilized nucleic acid molecule; and e) detecting the elongated product that is immobilized to the matrix. In some embodiments, the nucleic acid molecule is associated with a labelling agent that binds to an analyte in the sample or is a probe that binds to the analyte in the sample.

In some aspects, provided herein is a method for analyzing a sample, comprising: contacting the sample with (i) a matrix-forming material to embed the sample in a three-dimensional polymerized matrix and (ii) a nucleic acid molecule, wherein the nucleic acid molecule comprises a free 3′ priming region; contacting the sample with: (i) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the nucleic acid molecule is complementary to the 3′ toehold domain of the initial hairpin molecule, and (ii) a polymerase having strand displacement activity; incubating the sample under conditions for polymerization to produce an elongated product of the immobilized nucleic acid molecule; and detecting the elongated product that is immobilized to the matrix. In some embodiments, the nucleic acid molecule is associated with a labelling agent that binds to an analyte in the sample or is a probe that binds to the analyte in the sample.

In some aspects, provided herein is a method for analyzing a sample embedded in a three-dimensional polymerized matrix, comprising: a) contacting the sample with a capture agent that interacts with an analyte in the sample and immobilizing the capture agent or the analyte to the matrix; b) contacting the sample with a nucleic acid molecule which forms a complex with the analyte and/or the capture agent, wherein the nucleic acid molecule comprises a free 3′ priming region; c) contacting the sample with: (i) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the probe is complementary to the 3′ toehold domain of the initial hairpin molecule, and (ii) a polymerase having strand displacement activity; d) providing conditions for polymerization to produce an elongated product of the immobilized nucleic acid molecule; and e) detecting the elongated product that is immobilized to the matrix.

In some aspects, provided herein is a method for analyzing a sample, comprising: contacting the sample with (i) a matrix-forming material to embed the sample in a three-dimensional polymerized matrix and (ii) a capture agent that interacts with an analyte in the sample and immobilizing the capture agent to the matrix, and (iii) a nucleic acid molecule which forms a complex with the analyte and/or the capture agent, wherein the nucleic acid molecule comprises a free 3′ priming region; contacting the sample with: (i) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the probe is complementary to the 3′ toehold domain of the initial hairpin molecule, and (ii) a polymerase having strand displacement activity; d) providing conditions for polymerization to produce an elongated product of the immobilized nucleic acid molecule; and e) detecting the elongated product that is immobilized to the matrix. In some embodiments, the matrix forming material, the capture agent, and the nucleic acid molecule are contacted with the sample simultaneously. In some embodiments, the matrix forming material is contacted with the sample prior to contacting the capture agent and the nucleic acid molecule with the sample simultaneously. In some embodiments, the matrix forming material and the capture agent is contacted with the sample simultaneously (e.g., as a mixture) prior to contacting the nucleic acid molecule with the sample.

In any of the preceding embodiments, the incubating step can be performed under conditions for polymerization, strand displacement, and annealing.

In any of the preceding embodiments, the nucleic acid molecule can be associated with a labelling agent that binds the analyte and/or the capture agent.

In any of the preceding embodiments, the labelling agent can be an antibody. In some embodiments, the nucleic acid molecule is conjugated to the antibody.

In any of the preceding embodiments, the nucleic acid molecule can be a probe that hybridizes to the analyte or to a target nucleic acid sequence associated with the analyte.

In any of the preceding embodiments, the capture agent can be an antibody.

In any of the preceding embodiments, the capture agent can comprise an oligo dT sequence.

In any of the preceding embodiments, the nucleic acid molecule can comprise a target hybridization region that hybridizes to the analyte or to the target nucleic acid sequence associated with the analyte.

In any of the preceding embodiments, the nucleic acid molecule can be immobilized to the matrix at an attachment point, and the target hybridization region can be located between the attachment point and the free 3′ priming region.

In any of the preceding embodiments, the nucleic acid molecule can be immobilized to the matrix at an attachment point, and the target hybridization region can be located 5′ of the attachment point and the free 3′ priming region.

In any of the preceding embodiments, contacting of the sample with a nucleic acid molecule or labelling agent can further comprise washing the sample to remove unhybridized nucleic acid molecules or labelling agent.

In some aspects, provided herein is a method for analyzing a sample embedded in a three-dimensional polymerized matrix, comprising: a) immobilizing a nucleic acid molecule in the sample to the matrix, wherein the nucleic acid molecule is an endogenous nucleic acid molecule or a product thereof, wherein the nucleic acid molecule comprises a free 3′ priming region; b) contacting the sample with: (i) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the endogenous nucleic acid molecule or product thereof is complementary to the 3′ toehold domain of the initial hairpin molecule, and (ii) a polymerase having strand displacement activity; d) providing conditions for polymerization to produce an elongated product of the immobilized endogenous nucleic acid molecule or product thereof; and e) detecting the elongated product that is immobilized to the matrix.

In some embodiments, the immobilizing comprises contacting the sample with a capture agent that interacts with the endogenous nucleic acid molecule in the sample and immobilizing the capture agent to the matrix. In some embodiments, the immobilizing comprises immobilizing the 5′ end of the endogenous nucleic acid molecule or product thereof in the sample. In some embodiments, the endogenous nucleic acid molecule is a messenger RNA (mRNA) molecule, wherein the capture agent comprises an oligo dT priming sequence, and the immobilizing step further comprises generating a complementary DNA (cDNA) product using the mRNA as a template. In some embodiments, the nucleic acid molecule is the cDNA product, and the initial hairpin molecule is designed to hybridize to the 3′ end of the cDNA product. In some embodiments, the unpaired 3′ toehold domain of the initial hairpin molecule is complementary to a 5′ paired stem subdomain of the initial hairpin molecule.

In any of the preceding embodiments, the method can further comprise contacting the sample with a second hairpin molecule comprising: (i) an unpaired 3′ toehold domain, (ii) a paired stem domain formed by intramolecular nucleotide base pairing between a 3′ subdomain of the second hairpin molecule and a 5′ subdomain of the second hairpin molecule, and (iii) a loop domain, wherein the 3′ toehold domain of the second hairpin molecule is complementary to the 5′ subdomain of the initial hairpin molecule.

In any of the preceding embodiments, the method can further comprise contacting the sample with deoxyribonucleotide triphosphates (dNTPs).

In any of the preceding embodiments, the elongated product produced in (d) can comprise a repeating nucleotide sequence domain, wherein the repeating domain is complementary to the stem domain of the initial and/or second hairpin molecule.

In any of the preceding embodiments, the nucleic acid molecule or capture agent can be immobilized to the matrix via a functional moiety that can be covalently cross-linked, copolymerize with, or otherwise non-covalently bound to the matrix. In some embodiments, the functional moiety can comprise an amine, acrydite, alkyne, biotin, azide, or thiol.

In any of the preceding embodiments, the nucleic acid molecule can be immobilized to the matrix at an attachment point, wherein the nucleic acid molecule comprises a spacer region between the attachment point and the free 3′ priming region. In some embodiments, the spacer region is of sufficient length to allow production of the elongated product from the nucleic acid molecule attached to the matrix. In some embodiments, the spacer region comprises a nucleotide sequence.

In any of the preceding embodiments, the spacer region can comprise one or more repeats of the sequence of the free 3′ priming end.

In any of the preceding embodiments, the nucleotide sequence of the spacer region can be between 20 and 50 nucleotides in length.

In any of the preceding embodiments, the spacer region can comprise a spacer moiety (e.g., polyethylene glycol, a carbon spacer, or a photo-cleavable spacer).

In any of the preceding embodiments, the detecting can comprise: (i) contacting the sample with a detection probe, wherein the detection probe comprises a detectable moiety; wherein the detection probe hybridizes directly or indirectly to the elongated product; and (ii) detecting the detectable moiety of the detection probe, thereby detecting the elongated product. In some embodiments, the detection probe can hybridize to the repeating nucleotide sequence domain. In some embodiments, the detection probe can hybridize indirectly to the elongated product. In some embodiments, the detection probe hybridizes to a repeating sequence of a secondary PER concatemer that is hybridized to the elongated product. In some embodiments, the secondary PER concatemer can be formed in situ in the hydrogel matrix according to the methods described herein. In some embodiments, the secondary PER concatemer can be formed in vitro and added to the matrix pre-formed.

In any of the preceding embodiments, the method can comprise contacting the sample with a matrix-forming material and using the matrix-forming material to form the three-dimensional polymerized matrix. In some embodiments, the matrix is a hydrogel matrix. In some embodiments, the analyte or the endogenous nucleic acid molecule is contained within the biological sample, and the matrix-forming material is introduced into the biological sample. In some embodiments, the analyte is a nucleic acid analyte or a protein analyte. In some embodiments, the analyte is a nucleic acid analyte. In some embodiments, the nucleic acid is an RNA molecule. In some embodiments, the RNA molecule is an mRNA molecule.

In any of the preceding embodiments, the method can comprise contacting the sample with a plurality of initial hairpin molecules for targeting a plurality of analytes, wherein each analyte of the plurality of analytes is or is associated with a nucleic acid molecule comprising a free 3′ priming region, wherein each initial hairpin molecule of the plurality comprises (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, and wherein the 3′ toehold domain is complementary to the free 3′ priming region of an analyte or associated with an analyte of the plurality of analytes.

In some embodiments, the method comprises providing conditions for polymerization to produce elongated products of a plurality of immobilized nucleic acid molecules, wherein the plurality of immobilized nucleic acid molecules correspond to the plurality of analytes.

In some embodiments, the plurality of initial hairpin molecules each comprise a sequence that uniquely corresponds to an analyte of the plurality of analytes. In some embodiments, the unpaired 3′ toehold domain of an initial hairpin molecule of the plurality comprises a sequence that uniquely corresponds to an analyte of the plurality of analytes. In some embodiments, an initial hairpin molecule of the plurality comprises a barcode sequence that uniquely corresponds to an analyte of the plurality of analytes. In some embodiments, the elongated products produced from the plurality of immobilized nucleic acid molecules each comprise a repeating nucleotide sequence domain corresponding to an analyte of the plurality of analytes.

In some embodiments, the method comprises contacting the sample with a capture agent that interacts with an analyte of the plurality of analytes in the sample and immobilizing the capture agent to the matrix. In some embodiments, the capture agent interacts with multiple analytes of the plurality of analytes. In other embodiments, the method comprises contacting the sample with a plurality of capture agents that interact with a plurality of analytes in the sample and immobilizing the capture agents to the matrix, wherein each capture agent interacts with a different analyte or a different subset of analytes.

In any of the preceding embodiments, the biological sample can be a processed or cleared biological sample.

In any of the preceding embodiments, the biological sample can be a tissue sample. In some embodiments, the tissue sample is a tissue slice between about 1 μm and about 50 μm in thickness. In some embodiments, the tissue slice is between about 5 μm and about 35 μm in thickness.

In any of the preceding embodiments, the biological sample can be embedded in a hydrogel.

In some embodiments, provided herein is a kit comprising: a) a nucleic acid molecule, wherein the nucleic acid molecule comprises a free 3′ priming region. In some embodiments, the nucleic acid molecule is associated with a labelling agent that binds to an analyte in the sample or is a probe that binds to the analyte in the sample; b) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the nucleic acid molecule is complementary to the 3′ toehold domain of the initial hairpin molecule; and c) a matrix-forming material. In some embodiments, the nucleic acid molecule or labelling agent comprises a functional moiety for immobilization to the matrix. In some embodiments, the functional moiety is selected from the group consisting of an amine, acrydite, alkyne, biotin, azide, or thiol.

In some aspects, provided herein is a kit comprising a nucleic acid molecule, wherein the nucleic acid molecule comprises a free 3′ priming region; b) a capture agent that interacts with an analyte in the sample. In some embodiments, the nucleic acid molecule is associated with a labelling agent that binds to the analyte or capture agent in the sample or is a probe that binds to the analyte or capture agent in the sample; c) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the nucleic acid molecule is complementary to the 3′ toehold domain of the initial hairpin molecule; and d) a matrix-forming material. In some embodiments, the capture agent comprises a functional moiety for immobilization to the matrix. In some embodiments, the functional moiety is selected from the group consisting of an amine, acrydite, alkyne, biotin, azide, or thiol.

In any of the preceding embodiments, the kit can further comprise labeling agents for detecting a repeating sequence in the elongated product. In some embodiments, the labeling agents can be detectably labeled probes.

In any of the preceding embodiments, the repeating sequence can be a sequence complementary to the 3′ toehold domain of the hairpin molecule, a sequence of the 3′ subdomain of the initial hairpin molecule, or a sequence of the free 3′ priming region of the nucleic acid molecule. In some embodiments, the sequence of the 3′ toehold domain or 3′ subdomain of the hairpin molecule or of the free 3′ priming region of the nucleic acid molecule corresponds to the analyte in the sample.

In any of the preceding embodiments, the repeating sequence can be a barcode sequence comprised by the hairpin molecule. In some embodiments, the barcode sequence of the hairpin molecule corresponds to the analyte in the sample

### BRIEF DESCRIPTION OF THE DRAWINGS

FIG. 1 shows a schematic diagram of an exemplary method for analyzing a biological sample embedded in a matrix, comprising (1) immobilizing a nucleic acid molecule (PER primer) to the matrix, (2) contacting the sample with an initial hairpin molecule, (3) producing an elongated product in a primer exchange reaction, and (4) detecting the elongated product. Although the hairpin shown is designed to generate an elongated product comprising repeated sequences of the free 3′ priming region (a), it will be understood by one of ordinary skill in the art that alternate subdomain sequences can be used to generate any desired set of sequences in the elongated product. For simplicity, the figure depicts direct immobilization of the nucleic acid molecule to the matrix. However, the nucleic acid molecule can also be immobilized to the matrix indirectly, by interacting with one or more analytes, capture agents, and/or labelling agents in a complex that is immobilized to the matrix.

FIGS. 2A-2C depict exemplary embodiments of the methods disclosed herein, wherein the nucleic acid molecule (PER primer) is a probe that is directly immobilized to the matrix (FIG. 2A), or is associated with a labelling agent that is directly immobilized to the matrix (FIG. 2B). In FIG. 2C, a labelling agent is associated with an analyte that is directly immobilized to the matrix. In some embodiments, the nucleic acid molecule comprises an optional spacer region between an attachment point for immobilization to the matrix and the free 3′ priming region.

FIGS. 3A-3C show exemplary embodiments of the methods disclosed herein, wherein the sample is contacted with a capture agent that binds directly or indirectly to an analyte in the sample, wherein the capture agent is immobilized to the matrix, and the nucleic acid molecule (PER primer) forms a complex with the analyte and/or capture agent. The capture agent can be a nucleic acid probe (FIG. 3A) or an antibody conjugated to a reporter oligonucleotide (FIG. 3B). In FIG. 3C, the capture agent is associated with an analyte that is directly immobilized to the matrix.

FIGS. 4A-4B depict exemplary embodiments of the methods disclosed herein, wherein the nucleic acid molecule (PER primer) is an endogenous nucleic acid (FIG. 4A) or a product thereof (e.g., an amplification/extension product of an endogenous nucleic acid; FIG. 4B).

FIG. 5 shows the formation of elongation (e.g., PER) products in the matrix in the presence of hairpin molecules.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Existing methods for analyzing the identity and spatial localization of analytes in a sample are limited by the ability to amplify and detect signals associated with the analytes, by the positional stability of the analytes in the sample, and by the ability to penetrate the sample with probes or other reagents required for detection. Various primer exchange reaction (PER) and PER-based signal amplification methods have been described in Saka, S. K., et al. (2019). “Immuno-SABER enables highly multiplexed and amplified protein imaging in tissues”. Nat Biotechnol 37, 1080-1090; Kishi, J. Y. et al. (2019). “SABER amplifies FISH: enhanced multiplexed imaging of RNA and DNA in cells and tissues”. Nature methods, 16(6), 533-544; U.S. Pat. Pub. 20190106733; and U.S. Pat. Pub 20200362398, the contents of each of which are herein incorporated by reference in their entirety. The PER reaction results in the formation of concatemer (repeat) sequences. The PER concatemer has a first domain that is complimentary to the nucleic acid strand linked to the target-specific binding partner and a second domain comprising repeat sequences. The repeated sequence may be used for hybridization of secondary PER concatemers, thereby forming a branched structure for further signal amplification, or by detection probes that hybridize to the repeated sequence.

The ability to amplify signals by hybridization of detection probes to a PER concatemer would be particularly relevant in the context of thick tissues, which can suffer from high levels of autofluorescence, light scattering, and optical aberration that can make signal detection. However, the length of PER concatemers limits their ability to penetrate biological samples such as matrix-embedded tissue sections.

In view of these issues, there is a desire for alternative methods of performing an assay for detecting analytes (e.g., analytes embedded in a matrix, whereby the relative position of the analytes is preserved in the matrix). In some aspects, the methods disclosed herein allow for a primer exchange reaction to be performed in a matrix, wherein the PER primer is a nucleic acid molecule comprising a free 3′ priming end, and the PER primer is directly or indirectly immobilized in the matrix. In some aspects, immobilization of the PER primer to the matrix provides positional stability for the PER primer in the matrix.

In some aspects, the PER primer is targeted to an analyte in the sample by a target hybridization sequence that binds to the analyte, or by an associated labelling agent that binds the analyte in the sample. In some aspects, after binding directly or indirectly to an analyte, the PER primer is covalently attached to the matrix, for example, by copolymerization or cross-linking. In some aspects, a structurally stable and chemically stable three dimensional matrix of PER primers is formed in the sample. According to this aspect, the three-dimensional matrix of PER primers, wherein each PER primer can be used to identify the associated analyte based on the sequence of its free 3′ priming region, allows for prolonged information storage and read-out cycles. In some aspects, the analytes themselves are immobilized within the matrix by covalent or noncovalent bonding. In this manner, the analytes may be considered to be attached to the matrix, and the PER primer may be considered to be indirectly attached to the matrix. By being immobilized to the matrix, such as by covalent bonding or crosslinking, the expression level and spatial relationship of the original analytes is maintained based on the spatial relationship of the PER primers immobilized in the matrix. By being immobilized to the matrix, such as by covalent bonding or crosslinking, the PER primers are resistant to movement due to diffusion or mechanical stress. In some aspects, the PER primers are localized to the analytes and the location of the analyte can be preserved.

In some aspects, the methods provided herein enable immobilization of a PER primer to a matrix without interfering with a primer exchange reaction initiated using said PER primer. For example, in some embodiments, PER primers provided herein comprise a functional moiety for attachment to a matrix, a spacer region, and a 3′ priming region for initiating PER. In some aspects, the spacer region is a region between an attachment point between the primer and the matrix (e.g., an attachment point at a functional moiety) and the 3′ priming region. In some embodiments, the inclusion of a spacer region between the matrix attachment point and the free 3′ priming end increases the efficiency of the PER reaction. Without wishing to be bound by theory, the present inventors realized that in some embodiments of the methods disclosed herein, inclusion of a spacer region could reduce steric hindrance for the initiation of PER in the case of a PER primer immobilized to a matrix. In some cases, the spacer region provides distance between the primer and the matrix such that the enzyme is not inhibited by the matrix any functional moieties for attaching the primer to the matrix. Optionally, the spacer region can comprise a target hybridization sequence for binding a target sequence comprised by or associated with an analyte in the sample.

In some embodiments, a composition disclosed herein provides a hybridization complex comprising a nucleic acid molecule comprising a functional moiety for attachment to a matrix, a spacer region, and a free 3′ priming region hybridized to a 3′ toehold domain of a hairpin molecule. In some embodiments, the nucleic acid molecule comprises, from 5′ to 3′, the functional moiety, the spacer region, and the free 3′ priming region. In some embodiments, the spacer region comprises a target hybridization sequence. In some embodiments, the hybridization complex further comprises an analyte hybridized by the target hybridization sequence. Analytes that can be analyzed by the presently disclosed methods are described in greater detail in Section II.

Kits for performing any of the methods disclosed herein are also provided. In some embodiments, provided herein is a kit for analyzing a biological sample embedded in a matrix, the kit comprising one of the nucleic acid molecules (PER primers), capture agents, labelling agents, and/or hairpin molecules disclosed herein. In some embodiments, the kit further comprises a matrix-forming material.

### II. Samples, Analytes, and Target Sequences

A. Samples and Matrix Embedding

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides.

Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, FFPE can be performed prior to embedding the sample in a matrix. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis (e.g., prior to matrix-embedding or introduction of a matrix forming material), the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprise one or more post-fixing (also referred to as postfixation) steps. In some embodiments, one or more post-fixing step is performed after contacting a sample with a polynucleotide disclosed herein, e.g., a nucleic acid molecule comprising a free 3′ priming region for initiation a primer exchange reaction. In some embodiments, one or more post-fixing step is performed after a hybridization complex comprising the nucleic acid molecule and a target (and optionally, a capture agent) is formed in a sample. In some embodiments, one or more post-fixing step is performed prior to an in situ primer exchange reaction (PER) using the immobilized nucleic acid molecule as a PER primer disclosed herein.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences. In some embodiments, the reporter oligonucleotide comprises a free 3′ priming region that is used to initiate PER according to the methods described herein.

A post-fixing step may be performed using any suitable fixation reagent disclosed herein, for example, 3% (w/v) paraformaldehyde in DEPC-PBS.

### (iv) Matrix Embedding

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

Matrix forming materials include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions.

According to one aspect, a matrix-forming material can be introduced into a cell. The cells are fixed with formaldehyde and then immersed in ethanol to disrupt the lipid membrane. The matrix forming reagents are added to the sample and are allowed to permeate throughout the cell. A polymerization inducing catalyst, UV or functional cross-linkers are then added to allow the formation of a gel matrix. The un-incorporated material is washed out and any remaining functionally reactive group is quenched. Exemplary cells include any cell, human or otherwise, including diseased cells or healthy cells. Certain cells include human cells, non-human cells, human stem cells, mouse stem cells, primary cell lines, immortalized cell lines, primary and immortalized fibroblasts, HeLa cells and neurons.

According to one aspect, a matrix-forming material can be used to encapsulate a biological sample, such as a tissue sample. The formalin-fixed embedded tissues on glass slides are incubated with xylene and washed using ethanol to remove the embedding wax. They are then treated with Proteinase K to permeabilized the tissue. A polymerization inducing catalyst, UV or functional cross-linkers are then added to allow the formation of a gel matrix. The un-incorporated material is washed out and any remaining functionally reactive group is quenched.

According to one aspect, the matrix-forming material forms a three dimensional matrix including a plurality of analytes and/or nucleic acid molecules comprising a free 3′ priming region (PER primers) while maintaining the spatial relationship of the analytes and/or PER primers. In this aspect, the plurality of analytes and/or PER primers is immobilized within the matrix material. The plurality of analytes and/or PER primers may be immobilized within the matrix material by co-polymerization of the nucleic acids with the matrix-forming material. The plurality of analytes and/or PER primers may also be immobilized within the matrix material by crosslinking of the analytes and/or PER primers to the matrix material or otherwise cross-linking with the matrix-forming material. The plurality of nucleic acids may also be immobilized within the matrix by covalent attachment or through ligand-protein interaction to the matrix, or by binding of the analytes and/or PER primers to capture agents that are immobilized to the matrix.

According to one aspect, the matrix is sufficiently optically transparent or otherwise has optical properties suitable for deep three dimensional imaging for high throughput information readout, such as for detection of the elongated PER product using labeled probes (e.g., fluorescently labeled probes).

According to one aspect, the matrix is porous thereby allowing the introduction of reagents (e.g., reagents for a primer exchange reaction) into the matrix at the site of a nucleic acid molecule comprising a free 3′ priming region for extension of the nucleic acid molecule in a primer exchange reaction. Additional control over the molecular sieve size and density is achieved by adding additional cross-linkers such as functionalized polyethylene glycols. According to one aspect, the nucleic acid molecules, which may represent bits of information (e.g., an analyte targeted by the nucleic acid molecule, optionally via association with a labelling agent), are readily accessed by oligonucleotides, such as labeled oligonucleotide probes, primers, PER catalytic molecules, enzymes and other reagents with rapid kinetics.

According to one aspect, the three dimensional matrix including nucleic acids is porous. According to one aspect, the three dimensional matrix including nucleic acids is porous to the extent that amplification reagents (e.g., polymerase) and catalytic molecules (e.g., hairpin molecules) for the primer exchange reaction can diffuse or otherwise move through the matrix to contact nucleic acids and thereby amplify or extend nucleic acids under suitable conditions. Porosity can result from polymerization and/or crosslinking of molecules used to make the matrix material. The diffusion property within the gel matrix is largely a function of the pore size. The molecular sieve size is chosen to allow for rapid diffusion of enzymes, oligonucleotides, formamide and other buffers used for amplification and sequencing (>50-nm). The molecular sieve size is also chosen so that large DNA or RNA amplicons do not readily diffuse within the matrix (<500-nm). The porosity is controlled by changing the cross-linking density, the chain lengths and the percentage of co-polymerized branching monomers

In some embodiments, the biological sample is immobilized in the hydrogel via cross-linking of the polymer material that forms the hydrogel. Cross-linking can be performed chemically and/or photochemically, or alternatively by any other hydrogel-formation.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the content of which is herein incorporated by reference in its entirety.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015, the content of which is herein incorporated by reference in its entirety.

Isometric expansion can be performed by anchoring one or more components of a biological sample to a gel (e.g., directly or indirectly anchoring an analyte and/or PER primer to a gel), followed by gel formation, proteolysis, and swelling. In some embodiments, analytes in the sample, products of the analytes, and/or probes (including, but not limited to, capture agent probes and/or PER primers) associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the content of which is herein incorporated by reference in its entirety).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-crosslinking

In some embodiments, the biological sample is reversibly cross-linked prior to or during an in situ assay round. In some aspects, the analytes, polynucleotides and/or amplification or extension product (e.g., an amplicon targeted by a PER primer, or an elongated product of a primer exchange reaction) of an analyte or a probe bound thereto can be anchored to a polymer matrix. For example, the polymer matrix can be a hydrogel. In some embodiments, one or more of the polynucleotide probe(s) and/or amplification or extension product (e.g., an amplicon targeted by a PER primer, or an elongated product of a primer exchange reaction) thereof can be modified to contain functional groups that can be used as an anchoring site to attach the polynucleotide probes and/or amplification product to a polymer matrix. In some embodiments, a modified capture agent probe or primer comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081, 2010/0055733, and 2020/0071751 the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after capture agents, labelling agents, and/or PER primers are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., nucleic acid molecules comprising a free 3′ priming region for initiation a primer exchange reaction, also referred to as “PER primers” herein), endonucleases to fragment DNA, fragmentation buffer for DNA, DNA polymerase enzymes for a primer exchange reaction (e.g., phi29 DNA polymerases, Bst DNA polymerases, and Bsu DNA polymerase, large fragment), and dNTPs used to extend the nucleic acid molecule and generate an elongated product in the sample. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums). In some embodiments, the methods disclosed herein comprise clearing PER primers that do not bind (directly or indirectly) to a target analyte in the sample.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as PER primers, hairpin molecules, labelling agents, capture agents, and/or PER reagents) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the content of which is herein incorporated by reference in its entirety. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, a nucleic acid molecule comprising a free 3′ priming end (a PER primer) as disclosed herein can hybridize to a target nucleic acid (e.g., cDNA or RNA molecule, such as an mRNA) and ligated in a templated ligation reaction (e.g., RNA-templated ligation (RTL) or DNA-templated ligation (e.g., on cDNA)) in the sample to generate a ligated PER primer for analysis. In some aspects, when two or more analytes are analyzed, a first and second probe that is specific for (e.g., specifically hybridizes to) each RNA or cDNA analyte are used (e.g., a 5′ domain of the PER primer that is specific for the first analyte, and a 3′ domain of the PER primer that is specific for the second analyte). In some embodiments, templated ligation of the PER primer is required for immobilization of the free 3′ priming region to the matrix. For example, the PER primer can be immobilized to the matrix via a functional moiety comprised by the 5′ domain, wherein a templated ligation is required to link the 5′ domain of the PER primer to the 3′ domain of the PER primer comprising the free 3′ priming region.

In some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, the product is targeted by a PER primer (e.g., a PER primer hybridizes specifically to the product). In some embodiments, the ligated product is the PER primer (e.g., the PER primer is provided as one or more molecules, wherein ligation stabilizes hybridization of the PER primer to a target sequence and/or enables immobilization of the free 3′ priming region to the matrix) In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the content of which is herein incorporated by reference in its entirety). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific subcellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., PER primers, hairpin molecules, or probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte). In some embodiments, the specific binding partner may be coupled to a nucleic acid molecule comprising a free 3′ priming end, which may be used as a PER primer (e.g., to generate an elongated product comprising repeated sequences for hybridization of detection probes).

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein. In some embodiments, the analyte binding moiety barcode is the target sequence for a PER primer described herein. Alternatively, the analyte binding moiety barcode can be the free 3′ priming region that is used to initiate a primer exchange reaction.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides or PER primers to the labelling agents may be achieved through any of a variety of direct or indirect, covalent or non-covalent associations or attachments. In some embodiments, the oligonucleotide attached to a labelling agent comprises a sequence that can serve as a PER primer and can be used as a reporter (e.g., a barcode). In some embodiments, the oligonucleotide attached to a labelling agent comprises both a reporter sequence (e.g., a barcode) and a sequence serving as a PER primer. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, the content of which is herein incorporated by reference in its entirety. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the labelling agent, or to a PER primer comprising a free 3′ priming region that identifies the labelling agent. For instance, the labelling agent may be directly coupled (e.g., covalently bound) to a hybridization oligonucleotide (e.g., a PER primer) that comprises a sequence that hybridizes with a sequence of the reporter oligonucleotide. Hybridization of the hybridization oligonucleotide (e.g., a PER primer) to the reporter oligonucleotide couples the labelling agent to the reporter oligonucleotide. In some embodiments, the reporter oligonucleotides (e.g., PER primers) are releasable from the labelling agent, such as upon application of a stimulus. For example, the reporter oligonucleotide or PER primer may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence or a unique molecular identifier (UMI) sequence.

In some cases, the labelling agent can comprise a reporter oligonucleotide (e.g., a PER primer) and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide (e.g., PER primer).

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product thereof is analyzed (e.g., is targeted by a PER primer, allowing generation of an elongated nucleic acid molecule using the PER primer and one or more catalytic molecules in situ). In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product of a labelling agent (e.g., comprising a reporter oligonucleotide) that directly or indirectly binds to an analyte in the biological sample is analyzed using the primer exchange reactions disclosed herein, wherein the PER primer and/or product is immobilized in a matrix.

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto, such as one comprising a free 3′ priming region). The other molecule can be another endogenous molecule or another labelling agent such as a PER primer or other probe (e.g., capture agent). Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences for hybridization of a PER primer. In some embodiments, the PER primer or the generated product can hybridize to an L-shaped or U-shaped probe that is hybridized to an endogenous analyte, a product of an endogenous analyte, and/or labelling agent in the sample.

(b) Ligation

In some embodiments, a product of an endogenous analyte and/or a labelling agent is a ligation product. In some embodiments, the ligation product is formed between two or more molecules that together form a PER primer (e.g., a 5′ domain of a PER primer and a 3′ domain of a PER primer). In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides (e.g., the ligated PER primer) prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

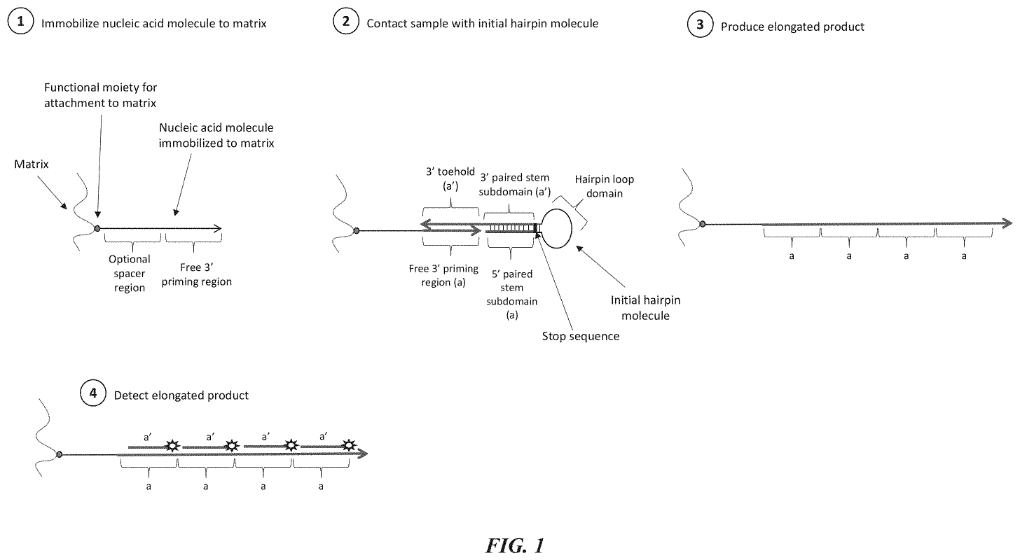

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a PER primer disclosed herein may be in a hybridization complex formed of a cellular nucleic acid in a sample and an exogenously added nucleic acid probe (e.g., a capture agent probe immobilized to a matrix). The exogenously added PER primer (depicted in FIGS. 1-4 as “nucleic acid molecule”) may comprise a free 3′ priming region that does not hybridize to the cellular nucleic acid but hybridizes to a 3′ toehold domain of a PER catalytic molecule (e.g., hairpin molecule as depicted in FIG. 1). In other examples, a product comprising a target sequence for a PER primer disclosed herein may be an extension or amplification product of an analyte or probe in the sample (e.g., an RCP generated using a circularizable probe or probe set which hybridizes to a cellular nucleic acid molecule such as genomic DNA or mRNA) and the PER primer (e.g., depicted in FIGS. 1-4 as “nucleic acid molecule”) may bind the extension or amplification product. The PER primer may comprise a free 3′ priming region that does not hybridize to the RCP but hybridizes to a PER catalytic molecule (e.g., a hairpin molecule). The PER primer may be optionally ligated to a cellular nucleic acid molecule or to a probe, e.g., an anchor probe that hybridizes to the extension or amplification product.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

C. Target Sequences

A target sequence for a probe disclosed herein may be comprised in any analyte disclose herein, including an endogenous analyte (e.g., a viral or cellular nucleic acid), a labelling agent, or a product or derivative of an endogenous analyte and/or a labelling agent. For example, a target sequence for a PER primer (e.g., depicted in FIGS. 1-4 as “nucleic acid molecule”) is comprised by a product generated by hybridizing a probe or probe set to an endogenous analyte and performing extension, ligation, and/or amplification using the probe or probe set.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos). In any of the preceding embodiments, the PER primer can be used as a probe to generate an elongated probe in situ in the matrix, providing repeating sequences (e.g., barcodes) for hybridization of detection probes. In any of the preceding embodiments, pre-formed PER concatemers can be added to the sample for use as probes.

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the elongated product of the primer exchange reaction are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and US 2021/0164039, which are hereby incorporated by reference in their entirety.

### III. Primer Exchange Reaction

In some aspects, provided herein are methods for analyzing a biological sample embedded in a matrix, comprising performing a primer exchange reaction using a nucleic acid molecule comprising a free 3′ priming region as a PER primer, wherein the nucleic acid molecule is directly or indirectly immobilized to a matrix. In some embodiments, the nucleic acid molecules is targeted to an analyte in the sample (e.g., by hybridizing to a target sequence comprised by or associated with the analyte, or by association with a labelling agent). In some embodiments, the immobilization of the PER primer and/or the analyte in the matrix preserves the relative spatial localization of the analyte in the sample, enabling detection and reconstruction of both the identities and three-dimensional positional information of analytes in the sample. The following section describes targeting and immobilization of the PER primer and the steps and components of the primer exchange reaction in the matrix.

(i) Targeting and Immobilizing PER primer

In some aspects, provided herein is a method for analyzing a sample embedded in a three-dimensional polymerized matrix, comprising: a) contacting the sample with a nucleic acid molecule (PER primer), wherein the nucleic acid molecule comprises a free 3′ priming region, and immobilizing the nucleic acid molecule to the matrix (e.g., directly or indirectly). The free 3′ priming region can be complementary to the 3′ toehold domain of a PER hairpin molecule, as shown in FIG. 1, thereby allowing the nucleic acid molecule to serve as a primer for the PER reaction. In some embodiments, the matrix is formed prior to or during the contacting of the sample with the PER primer.

In some embodiments, the nucleic acid molecule is directly immobilized to the matrix, e.g., via direct cross-linking or co-polymerization with the matrix. In some embodiments, the nucleic acid molecule is indirectly immobilized to the matrix, e.g., via direct or indirect hybridization to an endogenous analyte, a labelling agent, or a product of an endogenous analyte or labelling agent, wherein the endogenous analyte, labelling agent, or product is immobilized to the matrix. Analytes, labelling agents, and products of endogenous analytes or labelling agents are described in Section II above.

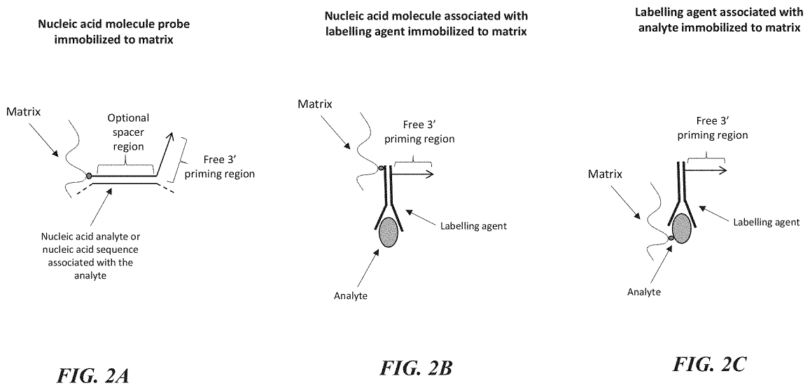

As shown in FIG. 2A, in some embodiments, the nucleic acid molecule is a nucleic acid probe that hybridizes to a target sequence in a nucleic acid analyte or a target sequence associated with an analyte (e.g., any of the target sequences described in Section II above). In some embodiments, the nucleic acid molecule comprises a target hybridization region, wherein the target hybridization region is complementary to a target sequence comprised by an analyte, labelling agent, or product in the sample. In some embodiments, the nucleic acid molecule is immobilized directly to the matrix, as shown in FIG. 2A. In other embodiments, the nucleic acid analyte or nucleic acid sequence associated with the analyte is immobilized directly or indirectly to the matrix, and the nucleic acid molecule is indirectly immobilized to the matrix by binding directly or indirectly to the analyte, labelling agent, or product (e.g., by hybridizing to the analyte, labelling agent, or product). In some embodiments, both the analyte and the nucleic acid molecule are directly immobilized to the matrix. In some embodiments, the sequence of the free 3′ priming region corresponds to the target hybridization sequence, whereby the target sequence can be identified by the sequence of the free 3′ priming region or a PER concatemer generated in a PER reaction using the free 3′ priming region and one or more hairpin molecules.

In some embodiments, the nucleic acid molecule is located 5′ of the free 3′ priming region. In some embodiments, the target hybridization region is at the 5′ end of the nucleic acid molecule. In some embodiments, the nucleic acid molecule is immobilized to the matrix at one or more attachment points, and the nucleic acid molecule optionally comprises a spacer region between the one or more attachment points and the free 3′ priming region, as shown in FIG. 2A. In some embodiments, the spacer region comprises the target hybridization region. In some embodiments, the spacer region further comprises one or more additional nucleotides that do not hybridize to the target sequence. In some embodiments, the additional nucleotides are located at the 3′ end of the spacer region.

In some embodiments, the nucleic acid molecule is associated with a labelling agent that binds to an analyte in the sample, wherein the labelling agent is directly or indirectly immobilized to the matrix. In some embodiments, the labelling agent may include an agent that interacts with an analyte (e.g., an endogenous analyte in a sample). As shown in FIG. 2B, in some embodiments, the nucleic acid molecule is directly conjugated to the labelling agent or hybridized to a reporter oligonucleotide conjugated to the labelling agent, wherein the reporter oligonucleotide is indicative of the analyte or portion thereof interacting with the labelling agent. In some embodiments, the labelling agent is directly immobilized to the matrix. For example, the labelling agent can be an antibody that is crosslinked to the matrix as shown in FIG. 2B, optionally via a functional crosslinkable moiety. Additionally or alternatively, the labelling agent can be indirectly immobilized to the matrix. For example, the analyte can be directly immobilized to the matrix as shown in FIG. 2C. In some embodiments, the nucleic acid molecule is fully complementary to the 3′ toehold domain of an initial hairpin molecule (e.g., there is no spacer region between the free 3′ priming region and the point at which the nucleic acid molecule is conjugated to a labelling agent). In some embodiments, the nucleic acid molecule comprises a spacer region between a 5′ end that is conjugated to a labelling agent and the 3′ priming end.

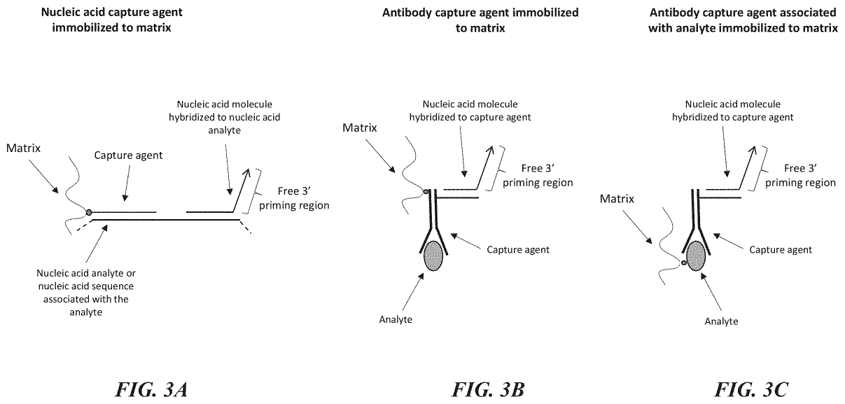

In some embodiments, provided herein is a method for analyzing a sample embedded in a three-dimensional polymerized matrix, comprising (a) contacting the sample with a capture agent that interacts with an analyte in the sample and immobilizing the capture agent to the matrix; and (b) contacting the sample with a nucleic acid molecule (PER primer) which forms a complex with the analyte and/or the capture agent as shown in FIGS. 3A-3C, wherein the nucleic acid molecule comprises a free 3′ priming region. The free 3′ priming region can be complementary to the 3′ toehold domain of a PER hairpin molecule, as shown in FIG. 1, thereby allowing the nucleic acid molecule to serve as a primer for the PER reaction.

In some embodiments, the capture agent is directly or indirectly immobilized to the matrix. Additionally or alternatively, the capture agent can be indirectly immobilized to the matrix via binding to the analyte. In some embodiments, the analyte is not directly immobilized to the matrix.

As shown in FIG. 3A, in some embodiments the capture agent is a nucleic acid probe that hybridizes to a nucleic acid analyte or nucleic acid sequence associated with an analyte. In some embodiments, the nucleic acid molecule also hybridizes to the nucleic acid analyte or nucleic acid sequence associated with the analyte, thereby forming a complex with the analyte and the capture agent, as shown in FIG. 3A. Although not depicted in FIG. 3A, it will be understood that the nucleic acid molecule can additionally or alternatively hybridize to the capture agent. For example, the capture agent can be an L-shaped probe comprising a region that hybridizes to a nucleic acid analyte and a region for hybridization to the nucleic acid molecule (PER primer). In some embodiments, a plurality of capture agents can be used to capture a plurality of different sequences. In some embodiments, a plurality of capture agents can be used to amplify a plurality of different sequences. In some instances, a plurality of capture agents may comprise the same spacer sequence or each capture agent of the plurality associated with a different analyte may be associated with a different spacer sequence.

In some embodiments, the capture agent is capable of binding a plurality of different analytes in the sample. For example, the capture agent can comprise an oligo dT sequence for binding to messenger RNA (mRNA) molecules in the sample, whereby the mRNA molecules are immobilized to the matrix via the capture agent. In some embodiments, the capture agent can comprise an oligo dT sequence and a crosslinkable moiety, such as a 5′ acrydite modification.

In some embodiments, the capture agent can be any suitable labelling agent capable of binding to an analyte in the sample. As shown in FIG. 3B, the capture agent can be an antibody that binds to an analyte in the sample. In some embodiments, the nucleic acid molecule can bind to an oligonucleotide molecule that is conjugated to the antibody, wherein the reporter oligonucleotide is indicative of the analyte or portion thereof interacting with the capture agent. In some embodiments, the nucleic acid molecule is associated with a labelling agent that binds the analyte and/or the capture agent. In some embodiments, the labelling agent is an antibody, optionally wherein the nucleic acid molecule is conjugated to the antibody. In some embodiments, the analyte bound by the capture agent can be directly immobilized to the matrix as shown in FIG. 3C.

In some embodiments, the nucleic acid molecule comprising a free 3′ priming region is an endogenous nucleic acid or a product thereof. In some embodiments, the method comprises contacting the sample with an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the endogenous nucleic acid molecule or product thereof is complementary to the 3′ toehold domain of the initial hairpin molecule. The 3′ toehold domain of the initial hairpin molecule can be designed to specifically hybridize to the free 3′ end of the endogenous nucleic acid molecule or product thereof, thereby allowing hybridization of the hairpin molecule and generation of a PER concatemer only in the presence of the endogenous nucleic acid molecule.

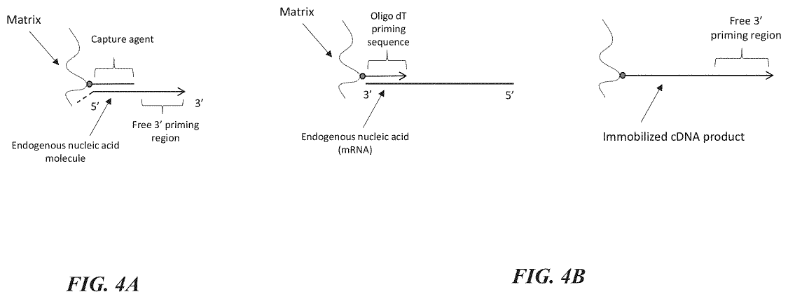

As shown in FIG. 4A, an endogenous nucleic acid molecule can be immobilized to the matrix via a capture agent (e.g., a capture probe) that hybridizes to a region of the endogenous nucleic acid molecule located 5′ to the free 3′ priming region. In some embodiments, the capture probe hybridizes to a region at the 5′ end of the endogenous nucleic acid molecule. In some embodiments, the endogenous nucleic molecule comprises a free 3′ priming region that specifically identifies the endogenous nucleic acid molecule. In some embodiments, the endogenous nucleic acid molecule does not comprise a poly A tail.

In some embodiments, the nucleic acid molecule comprising a free 3′ priming region is a product of an endogenous nucleic acid molecule. For example, as shown in FIG. 4B, the nucleic acid molecule can be a polymerase extension product generated using the endogenous nucleic acid molecule as a template. In some embodiments, the product of the endogenous nucleic acid molecule is generated using a primer that is immobilized to the matrix, whereby the product is immobilized to the matrix. In some embodiments, the primer comprises a functional moiety (e.g., a 5′ crosslinkable moiety) that is immobilized to the matrix. In a specific example shown in FIG. 4B, the primer can comprise an oligo dT priming sequence that hybridizes to a polyA tail of a messenger RNA molecule in the sample to prime an extension reaction, thereby generating a cDNA product. In some embodiments, the oligo dT primer comprises a 5′ acrydite moiety for immobilization to the matrix, whereby the cDNA product is immobilized to the matrix at its 5′ end. In some embodiments, the RNA template is removed by a wash step or by digestion with RNase H, thereby freeing the 3′ priming region of the cDNA.

Exemplary methods of making a three-dimensional matrix and immobilizing target analytes in the matrix are provided in U.S. Pat. Nos. 10,138,509 and 10,266,888, the contents of which are herein incorporated by reference in their entirety. In some embodiments, the methods disclosed herein include making a three-dimensional matrix including analytes and/or nucleic acid molecules (PER primers) covalently bound into a matrix or into or to a matrix material. The analytes, capture agents, and/or nucleic acid molecules may be co-polymerized with the matrix material or cross-linked to the matrix material or both. In some embodiments, the analytes, capture agents, and/or nucleic acid molecules are non-covalently bound to the matrix, e.g., via a ligand-ligand binding pair. According to one aspect, analytes, capture agents, and/or nucleic acid molecules are covalently attached to a matrix material to preserve their spatial orientation in the x, y and z axes within the matrix. It is to be understood that the three-dimensional matrix may include a matrix material and that the term copolymer, matrix and matrix material may be used interchangeably. Useful methods also include immobilizing endogenous analytes (e.g., protein or nucleic acid analytes) within their native environment, such as within a cell or within a tissue sample. The three-dimensional nucleic acid matrix can be generated in situ in a cell or tissue sample to preserve the naturally occurring analytes and their spatial orientation in cells, tissues or any other complex biomaterial. According to this aspect, the location of the analytes and their relative position is identified as a three-dimensional structure, such as within subcellular compartments, within cells, within tissues, as three dimensional nucleic acid assemblies, as three dimensional nucleic acid material, etc. The analytes can be detected using a primer exchange reaction (PER) in situ wherein the nucleic acid molecule for initiating the PER is directly or indirectly immobilized to the matrix, thereby providing positional information of an analyte bound by the nucleic acid molecule within the cell or tissue.

In some aspects of the methods disclosed herein, the nucleic acid molecule or capture agent is immobilized to the matrix via a functional moiety that can be covalently cross-linked, copolymerize with, or otherwise non-covalently bound to the matrix (e.g., as shown in FIG. 1). In some embodiments, the functional moiety comprises an amine, acrydite, alkyne, biotin, azide, or thiol. One or more nucleotides of a nucleic acid molecule (PER primer) or capture agent may be modified to include a functional moiety for attachment to the matrix. The functional moiety may be covalently attached to the matrix within the sample, covalently cross-linked, copolymerized with or otherwise non-covalently bound to the matrix. A functional moiety may be activatable. In some embodiments, an activatable functional moiety is inert (e.g., does not bind a target) until activated (e.g., by exposure of the activatable functional moiety to light, heat, one or more chemical compounds or the like). In some embodiments, an analyte (e.g., a protein analyte) or labelling agent (e.g., an antibody) can comprise a functional moiety for attachment to the matrix.

The functional moiety can react with a cross-linker. The functional moiety can be part of a ligand-ligand binding pair. An exemplary functional moiety includes an amine, amine reactive groups, acrydite, an acrydite modified entity, alkyne, biotin, azide, thiol, and a thiol-modified entity and entities suitable for click chemistry techniques. Biotin, or a derivative thereof, may be used as a matrix attachment moiety when the matrix includes an avidin/streptavidin derivative or an anti-biotin antibody (e.g., a detectably labelled antibody). In some embodiments, a polyacrylamide gel matrix is co-polymerized with acrydite-modified streptavidin. In some instances, biotinylated nucleic acid molecules or capture agents is captured after the matrix is formed. In one example, a polyacrylamide gel matrix is co-polymerized with acrydite-modified streptavidin monomers and biotinylated nucleic acid molecules or capture agents, using a suitable acrylamide:bis-acrylamide ratio to control the cross-linking density. Digoxigenin may be used as a matrix attachment moiety and subsequently bound by an anti-digoxigenin antibody attached to the matrix. An aminoallyl-dUTP residue may be incorporated into an oligonucleotide and subsequently coupled to an N-hydroxy succinimide which may be incorporated into the matrix. In some embodiments, a Dibenzocyclooctyne (DBCO)-azide can be used for matrix attachment. In some embodiments, a DBCO moiety is incorporated into the oligonucleotide, and the matrix comprises an azide. In some embodiments, the DBCO is reacted with the azide in a strain promoted alkyne-azide cycloaddition (SPAAC). In some embodiments, an analyte is attached to the matrix using acrydite (e.g., by copolymerization), NHS ester (e.g., coupling of an NHS ester linked to the analyte with an amine on the matrix), DBCO (e.g., coupling a DBCO in the analyte to an azide on the matrix), sulfhydryl (e.g, coupling a sulfhydryl in or associate with the analyte to a maleimide on matrix), amine (e.g., coupling an amine in or associated with the analyte to a carboxyl on the matrix, or coupling a carboxyl in or associated with the analyte to an amine on the matrix). In general, any member of a conjugate pair or reactive pair may be used to attach a nucleotide, whether in an oligonucleotide or a chromosome, to a matrix.

Functional moieties for attachment to a matrix include chemical cross-linking agents. Cross-linking agents typically contain at least two reactive groups that are reactive towards numerous groups, including, but not limited to, sulfhydryls and amines, and create chemical covalent bonds between two or more molecules. Functional moieties include cross-linking agents such as primary amines, carboxyls, sulfhydryls, carbohydrates and carboxylic acids. Protein molecules have many of these functional groups and therefore proteins and peptides can be readily conjugated using cross-linking agents. Cross-linking agents are commercially available (Thermo Scientific (Rockford, Ill.)). In the case of crosslinking, the matrix attachment moiety may be cross-linked to modified dNTP or dUTP or both. Suitable exemplary cross-linker reactive groups include imidoester (DMP), succinimide ester (NETS), maleimide (Sulfo-SMCC), carbodiimide (DCC, EDC) and phenyl azide. Cross-linkers within the scope of the present disclosure may include a spacer moiety. Such spacer moieties may be functionalized. Such spacer moieties may be chemically stable. Suitable exemplary spacer moieties include polyethylene glycol, carbon spacers, cleavable (e.g., photo-cleavable or chemically cleavable) spacers and other spacers and the like.

According to certain aspects, a three dimensional nucleic acid matrix is provided wherein a plurality of nucleic acid molecules and/or analytes are immobilized, such as by covalent bonding to the matrix, in a three dimensional space relative to one another. In this context, the nucleic acid molecules for initiating PER are rigidly fixed to the extent that they maintain their coordinate position within the matrix. It is to be understood that even though a nucleic acid molecule may be covalently attached to the three dimensional matrix material, the nucleic acid molecule itself may be capable of movement though bound to the matrix, such as for example, when a nucleic acid sequence is bound to the matrix at a single location on the nucleic acid.

In some embodiments, the nucleic acid molecule comprises a spacer region between the free 3′ priming region and one or more attachment points at which the nucleic acid molecule is immobilized to the matrix. In some embodiments, the spacer region is of sufficient length to allow production of the elongated product from the nucleic acid molecule attached to the matrix. In some embodiments, the spacer region comprises a nucleotide sequence. Optionally, the nucleotide sequence of the spacer is between 1 and 50 nucleotides in length, such as between 5 and 50, between 10 and 50, between 20 and 50, between 10 and 40, between 10 and 30, or between 20 and 30 nucleotides in length. In some embodiments, the spacer region comprises one or more repeats of the sequence of the free 3′ priming end. In some embodiments, the spacer region comprises a target hybridization region that is complementary to a target sequence comprised by or associated with an analyte. In some embodiments, the spacer region comprises a non-nucleic acid spacer. In some embodiments, the spacer region comprises a spacer moiety (e.g., polyethylene glycol, a carbon spacer, a chemical cleavable spacer or a photo-cleavable spacer). In some embodiments, the spacer comprises a chemically cleavable disulfide bond.

A nucleic acid molecule comprising a free 3′ priming region, in some embodiments, includes an unpaired 5′ target hybridization region that binds to a target sequence (e.g., a sequence comprised by or associated with an analyte) and an unpaired (“free”) 3′ primer region that binds to the unpaired 3′ toehold domain of a catalytic molecule to initiate a primer exchange reaction. In some embodiments, the nucleic acid molecule is immobilized to the matrix at one or more attachment points located 5′ to the free 3′ primer region. A nucleic acid molecule comprising a free 3′ priming region, in some embodiments, is comprised of DNA, RNA or a combination of DNA and RNA. In a PER reaction (see nucleic acid molecule in FIG. 1 and as an illustrative example), the free 3′ priming region of the nucleic acid molecule binds to the toehold domain of a catalytic molecule (e.g., a hairpin molecule) and extension of the nucleic acid molecule by a strand displacement polymerase present in the reaction solution displaces one of the subdomains of the paired stem domain of the catalytic molecule through a branch migration process. The overall effect is that the 5′ subdomain of the hairpin paired stem domain is replaced with the extended (newly synthesized) free 3′ priming region.

In some embodiments, a nucleic acid molecule comprising a free 3′ priming region (e.g., comprising a target hybridization region and free 3′ priming region, or comprising a free 3′ priming region associated with a labelling agent such as an antibody) has a length of 10-100 nucleotides. For example a nucleic acid molecule comprising a free 3′ priming region may have a length of 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, 45-50, 50-60, 60-70, 70-80, 80-90, or 90-100 nucleotides. In some embodiments, a nucleic acid molecule comprising a free 3′ priming region has a length of 10, 15, 20, 25, 30, 35, 40, 45 or 50 nucleotides. In some embodiments, a nucleic acid molecule comprising a free 3′ priming region has a length of 15, 16, 17, 18, 19, 20, 21, 22, 23, 24 or 25 nucleotides. A nucleic acid molecule comprising a free 3′ priming region, in some embodiments, is longer than 50 or 100 nucleotides, or shorter than 10 nucleotides.

In some embodiments, a free 3′ priming region (the nucleotide sequence that binds to the toehold domain of a catalytic molecule) has a length of 10-30 nucleotides. For example, a free 3′ priming region may have a length of 10-25, 10-20, 10-15, 15-30, 15-25, 15-20, 20-30, 20-25, or 25-30, nucleotides. In some embodiments, a free 3′ priming region has a length of 10, 15, 20, 25, or 30 nucleotides. In some embodiments, a free 3′ priming region has a length of 15, 16, 17, 18, 19, 20, 21, 22, 23, 24 or 25 nucleotides.

In some embodiments, a nucleic acid molecule comprising a free 3′ priming region further comprises a spacer region between an attachment point at which the nucleic acid molecule is immobilized to the matrix and the free 3′ priming region (e.g., between one or more functional moieties for attachment to the matrix and the free 3′ priming region). In some embodiments, the spacer region comprises a non In some embodiments, the spacer region has a length of 10-30 nucleotides. For example, a spacer region may have a length of 10-25, 10-20, 10-15, 15-30, 15-25, 15-20, 20-30, 20-25, or 25-30, nucleotides. In some embodiments, a spacer region has a length of 10, 15, 20, 25, or 30 nucleotides. In some embodiments, a spacer has a length of 15, 16, 17, 18, 19, 20, 21, 22, 23, 24 or 25 nucleotides. In some embodiments, the spacer region is or comprises a target hybridization region for binding to a target sequence. In some embodiments, the spacer region does not comprise a target hybridization region.

In some embodiments, the PER primer (depicted in FIGS. 1-4 as “nucleic acid molecule”) may comprise a region of a repeated sequence. In some cases, the PER primer comprises a poly T spacer region with have a length of 5-25, 5-20, 5-15, 5-30, 10-25, 10-20, 10-30 nucleotides of T's. In some embodiments, the spacer region may reduce steric hindrance for the initiation of PER when a PER primer is immobilized to a matrix (e.g., a functional moiety for immobilization and the enzyme for PER elongation). In some examples, the spacer of about 10 or more nucleotides is sufficient to avoid hindrance. In some cases, the PER primer comprises a region of a repeated sequence that is 10 nucleotides long repeated at least 1, 2, 3, 4, 5 times.

In some embodiments, the nucleic acid molecule comprising a free 3′ priming region for initiating a primer exchange reaction is contacted with the sample within a suitable concentration range for hybridization to a target sequence in the sample. In some embodiments, the nucleic acid molecule is provided in a concentration from 10 nM to 1000 nM. In some embodiments, the concentration of nucleic acid molecule with a 3′ priming region in a primer exchange reaction is 10-20, 10-30, 10-40, 10-50, 10-60, 10-70, 10-80, 10-90, 10-100, 10-125, 10-150, 10-200, 25-50, 25-75, 25-100, 25-150, 25-200, 50-75, 50-100, 50-150 or 50-200 nM. In some embodiments, the e concentration of nucleic acid molecule with a 3′ priming region in a primer exchange reaction is 100-200, 100-300, 100-400, 100-500, 100-600, 100-70, 100-800, 100-900 or 100-1000 nM. In some embodiments, e concentration of nucleic acid molecule with a 3′ priming region in a primer exchange reaction is 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 nM. In some embodiments, the e concentration of nucleic acid molecule with a 3′ priming region in a primer exchange reaction is 100, 200, 300, 400, 500, 600, 700, 800, 900 or 1000 nM. The concentration of nucleic acid molecule with a 3′ priming region in a primer exchange reaction may be less than 10 nM or greater than 1000 nM.

(ii) PER components and steps

In some aspects of the methods disclosed herein, the method comprises contacting the sample with one or more catalytic hairpin molecules to perform a primer exchange reaction in situ in the matrix-embedded sample. In some embodiments, the method comprises contacting the sample with: (i) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the nucleic acid molecule is complementary to the 3′ toehold domain of the initial hairpin molecule, and (ii) a polymerase having strand displacement activity. In some embodiments, the hairpin molecule is provided in a reaction mixture together with the polymerase, optionally wherein the reaction mixture further comprises dNTPs and a suitable reaction buffer. In some embodiments, the method further comprises providing conditions for polymerization to produce an elongated product of the immobilized endogenous nucleic acid molecule or product thereof in the matrix-embedded sample; and e) detecting the elongated product that is immobilized to the matrix.

In some embodiments, the unpaired 3′ toehold domain of the initial hairpin molecule is complementary to a 5′ paired stem subdomain of the initial hairpin molecule, as shown in FIG. 1. For example, the hairpin molecule can comprise, from 5′ to 3′, a 5′ paired stem subdomain of sequence “a”, a loop domain, a 3′ paired stem subdomain of sequence “a”′, wherein a′ is complementary to a, and a 3′ toehold region of sequence a′, wherein the 3′ toehold region is complementary to the free 3′ priming region of the nucleic acid molecule (sequence a). In this embodiment, the 3′ toehold region is a repeat of the sequence of the 3′ paired stem subdomain. This catalytic hairpin design can be used to generate an elongated product that is a concatemer of repeating sequences “a”, complementary to the repeat sequences of the 3′ toehold region and the 3′ paired stem subdomain. In some embodiments, the repeating sequences of the elongated product are repeated barcode sequences corresponding to the analyte in the sample, whereby the analyte can be identified by detecting the barcode using one or more labeled probes (e.g., decoding the barcode sequence via sequential hybridization of labeled probes). The same catalytic hairpin molecule can be used to repeatedly extend the product in the primer exchange reaction.

In some embodiments, the hairpin molecule comprises a sequence that corresponds to the analyte in the sample, whereby the elongated primer comprises a sequence corresponding the analyte. In some embodiments, the hairpin molecule comprises a barcode sequence corresponding to the analyte in the sample. In some embodiments, the barcode sequence is comprised by the 3′ toehold region and/or the 3′ paired stem. In some embodiments, the nucleic acid molecule (PER primer) comprises a sequence that corresponds to the analyte in the sample. In some embodiments, the free 3′ priming region of the nucleic acid molecule comprises a sequence (e.g., a barcode sequence) that corresponds to the analyte in the sample.

In some embodiments, the method can further comprise contacting the sample with a second hairpin molecule comprising: (i) an unpaired 3′ toehold domain, (ii) a paired stem domain formed by intramolecular nucleotide base pairing between a 3′ subdomain of the second hairpin molecule and a 5′ subdomain of the second hairpin molecule, and (iii) a loop domain, wherein the 3′ toehold domain of the second hairpin molecule is complementary to the 5′ subdomain of the initial hairpin molecule. Additional hairpin molecules can be designed in this manner to grow an elongated product comprising multiple specified sequences.

In some embodiments, any of the elongated products described herein can comprise a repeating nucleotide sequence domain, wherein the repeating domain is complementary to the stem domain of the initial and/or second hairpin molecule.

A paired domain (considered a “stem domain” with reference to hairpins) of a catalytic molecule comprised of nucleic acid refers to a paired sequence of nucleotides (e.g., Watson-Crick nucleobase pairing) located 5′ from (and, in some embodiments, directly adjacent to) the unpaired toehold domain of a catalytic molecule, as shown in FIG. 1. The paired domain of a catalytic molecule is formed by nucleotide base pairing between a “displacement strand” and a “template strand” containing a toehold domain. The paired domain (stem domain) of a catalytic hairpin molecule is formed by intramolecular base pairing (base pairing between nucleotides within the same molecule) of two subdomains of a catalytic hairpin molecule: e.g., an internal/central subdomain located 5′ from the toehold domain bound (hybridized) to a subdomain located at the 5′ end of the catalytic hairpin. The length of a paired domain of a catalytic molecule comprised of nucleic acid may vary. In some embodiments, a paired domain has a length of 5-40 nucleotides. For example, a paired domain may have a length of 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a paired domain has a length of 5, 10, 15, 20, 25, 30, 35 or 40 nucleotides. In some embodiments, a paired domain has a length of 10, 11, 12, 13, 14, 15, 16, 17, 18, 19, 20, 21, 22, 23, 24 or 25 nucleotides. A paired domain, in some embodiments, is longer than 40 nucleotides, or shorter than 5 nucleotides.

While a paired domain is generally formed by intramolecular base pairing of two subdomains of a catalytic molecule, it should be understood that this paired domain may contain at least one mismatch pair (e.g., pairing of A with C or G, or pairing of T with C or G). In some embodiments, the paired domain has 1-5 mismatch nucleotide base pairs. For example, a paired domain may have 1, 2, 3, 4 or 5 mismatch nucleotide base pairs.

A catalytic molecule generally includes an unpaired (single-stranded) 3′ toehold domain and a paired (double-stranded) domain 5′ from (and, in some embodiments, directly adjacent to) the 3′ toehold domain. A catalytic molecule may be comprised of DNA, RNA or a combination of DNA and RNA. Catalytic hairpin molecules further include a loop domain at the end of the molecule opposite to the 3′ toehold domain. The kinetics of primer exchange reactions can be controlled by modifying the length, composition and concentration of the catalytic molecules (e.g., one or more domains of the catalytic molecules), for example.

A catalytic hairpin molecule (see FIG. 1 as an illustrative example) includes a 3′ toehold domain linked to a paired stem domain (e.g., formed by intramolecular binding of a 5′ paired stem subdomain “a” to a 3′ paired stem subdomain “a′”) linked to a hairpin loop domain (loop-like structure). Thus, in some embodiments, a catalytic hairpin molecule comprises a single nucleic acid strand formed into a hairpin structure through intramolecular base pairing. Catalytic molecules without a loop domain (“duplexes”) are also provided herein. The length of a catalytic molecule (e.g., catalytic hairpin molecule) may vary. In some embodiments, a catalytic molecule comprised of nucleic acid has a length (5′ to 3′) of 25-300 nucleotides. For example, a catalytic molecule comprised of nucleic acid may have a length of 25-250, 25-200, 25-150, 25-100, 25-50, 50-300, 50-250, 50-200, 50-150 or 50-100 nucleotides. In some embodiments, a catalytic molecule comprised of nucleic acid has a length of 30-50, 40-60, 50-70, 60-80, 70-90, 80-100, 100-125, 100-150 or 100-200 nucleotides. In some embodiments, a catalytic molecule comprised of nucleic acid has a length of 35, 36, 37, 38, 39, 40, 41, 42, 43, 44, 45, 46, 47, 48, 49. 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. A catalytic molecule comprised of nucleic acid, in some embodiments, is longer than 300 nucleotides, or shorter than 25 nucleotides.

A toehold domain is an unpaired domain located at the 3′ end of the catalytic hairpin molecule (an unpaired 3′ domain) and binds to a primer domain of a probe strand. In some embodiments, a toehold domain (and thus the catalytic molecule) comprise a nucleotide sequence complementary to (wholly or partially, e.g., in length and/or nucleotide composition) a primer domain of a probe strand. In some embodiments, the toehold domain nucleotide sequence is longer or shorter than the free 3′ priming region of the nucleic acid molecule. In other embodiments, the toehold domain nucleotide sequence is the same length as the free 3′ priming region of the nucleic acid molecule. The length of a toehold domain may vary. In some embodiments, a toehold domain has a length of 5-40 nucleotides. For example, a toehold domain may have a length of 2-35, 2-30, 2-25, 2-20, 2-15, 2-10, 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a toehold domain has a length of 5, 10, 15, 20, 25, 30, 35 or 40 nucleotides. In some embodiments, a toehold domain has a length of 10, 11, 12, 13, 14, 15, 16, 17, 18, 19, 20, 21, 22, 23, 24 or 25 nucleotides. A toehold domain, in some embodiments, is longer than 40 nucleotides, or shorter than 5 nucleotides.

A loop domain of a catalytic hairpin molecule refers to a primarily unpaired sequence of nucleotides that form a loop-like structure at the end (adjacent to) of the stem domain (opposite the 3′ toehold domain). The length of a loop domain may vary. In some embodiments, a loop domain of a catalytic hairpin molecule comprised of nucleic acid has a length 3-200 nucleotides. For example, a loop domain may have a length of 3-175, 3-150, 3-125, 3-100, 3-75, 3-50, 3-25, 4-175, 4-150, 4-125, 4-100, 4-75, 4-50, 4-25, 5-175, 5-150, 5-125, 5-100, 5-75, 5-50 or 5-25 nucleotides. In some embodiments, a loop domain has a length of 3-10, 3-15, 32-10, 3-25, 3-30, 3-35, 3-40, 3-35, 3-40, 3-45, 3-50, 4-10, 4-15, 4-10, 4-25, 4-30, 4-35, 4-40, 4-35, 4-40, 4-45 or 4-50 nucleotides. In some embodiments, a loop domain has a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49 or 50 nucleotides. A loop domain, in some embodiments, is longer than 300 nucleotides.

In some embodiments, a catalytic molecule does not contain a hairpin loop domain. For example, a catalytic molecule may simply be a duplex comprising a 3′ unpaired toehold domain adjacent to a paired domain, similar to a stem domain (without the adjacent loop domain). Catalytic molecules that do not include a loop domain may be stabilized at the end opposite the 3′ toehold domain through crosslinking or nucleotide base complementarity between a stretch (e.g., 10 or more) nucleotide base pairs.

In some embodiments, each catalytic molecule is a catalytic hairpin molecule further comprising a loop domain located between the first and second domains. In some embodiments, each catalytic hairpin molecule is comprised of a single strand of DNA having a length of 25-300 nucleotides. In some embodiments, the catalytic molecule comprises two strands of DNA bound together, whereby the first strand contains the first domain, and the second strand comprises the second and third domains.

In some embodiments, each catalytic molecule further comprises a stopper molecule or modification that terminates polymerization located between the first and second domains of the same catalytic molecule (e.g., a stop sequence, as shown in FIG. 1). For example, the molecule or modification that terminates polymerization may be selected from a triethylene glycol (TEG), 18-atom hexa-ethylene glycol, adenylation, azide, digoxigenin, cholesteryl-TEG, 3-cyanovinylcarbazole (CNVK), iso-dG and iso-dC. In some embodiments, the stop sequence is guanine and the catalytic molecule is comprised of adenine, thymine and cytosine, or in other embodiments, the stop sequence is cytosine and the catalytic molecule is comprised of adenine, thymine and guanine.

In some aspects of the methods disclosed herein, the method comprises contacting the sample with a pre-formed PER concatemer. In some aspects, the pre-formed PER-concatemer is contacted with the sample prior to or concurrently with a matrix forming material. In some embodiments, the pre-formed PER concatemer is contacted with the sample embedded in a matrix (e.g., after matrix formation). In some embodiments, the pre-formed PER concatemer is associated with a labelling agent that binds to an analyte in the sample or is a probe that binds to the analyte in the sample. In some embodiments, the pre-formed PER concatemer forms a complex with an analyte and/or with a capture agent that interacts with the analyte in the sample, wherein the analyte and/or capture agent is directly or indirectly immobilized to the matrix. In some embodiments, the pre-formed PER concatemer hybridizes to a PER concatemer that is generated in situ in the matrix-embedded sample according to any of the methods disclosed herein.

In some embodiments of the methods provided herein, the method comprises washing the sample to remove unhybridized nucleic acid molecules (e.g., unhybridized PER primers) or labelling agents. In some embodiments, the washing step is performed prior to immobilization of the nucleic acid molecule in the sample (e.g., prior to cross-linking or co-polymerization with a matrix forming material). In some embodiments, the wash is performed under stringent conditions.

According to one aspect, the three dimensional matrix material is chemically inert and thermally stable to allow for various reaction conditions and reaction temperatures. According to this aspect, the three dimensional matrix material is chemically inert and thermally stable to conditions used in amplification and sequencing methods. In some embodiments, the matrix is chemically inert and stable to allow for conditions for a primer exchange reaction.

In some embodiments, the matrix-embedded sample is incubated under conditions for polymerization (e.g., using a strand-displacing polymerase) to generate an elongated product from the nucleic acid molecule and catalytic molecules (e.g., hairpin molecules). In some embodiments, the strand-displacing polymerase is selected from phi29 DNA polymerases, Bst DNA polymerases, and Bsu DNA polymerase, large fragment. In some embodiments, a reaction mixture comprises aqueous buffer, optionally phosphate buffered saline (PBS). In some embodiments, a reaction mixture comprises MgSO₄, optionally at a concentration of 5-50 mM.

The concentration of catalytic molecules (e.g., catalytic hairpins) in a primer exchange reaction may be, for example, 5 nM to 1000 nM. In some embodiments, the catalytic molecule concentration in a multiplexed primer exchange reaction is 5-10, 5-20, 5-30, 5-40, 5-50, 5-60, 5-70, 5-80, 5-90, 5-100, 5-125, 5-150, 5-200, 10-50, 10-75, 10-100, 10-150, 10-200, 25-75, 25-100, 25-125 or 25-200 nM. In some embodiments, the catalytic molecule concentration in a multiplexed primer exchange reaction is 10-200, 10-300, 10-400, 10-500, 10-600, 10-70, 10-800, 10-900 or 10-100 nM. In some embodiments, the catalytic molecule concentration in a multiplexed primer exchange reaction is 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 nM. In some embodiments, the catalytic molecule concentration in a multiplexed primer exchange reaction is 10, 20, 30, 40, 50, 60, 70, 80, 90 or 100 nM. The concentration of catalytic molecule in a multiplexed primer exchange reaction may be less than 5 nM or greater than 1000 nM.

The ratio of primer to catalytic molecule in multiplexed primer exchange reaction may be 2:1 to 100:1. In some embodiments, the ratio of primer to catalytic molecule is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1. In some embodiments, the ratio of primer to catalytic molecule is 30:1, 40:1, 50:1, 60:1, 70:1, 80:1 or 90:1.

The number of different catalytic molecules in a multiplexed primer exchange reaction is non-limiting. A multiplexed primer exchange reaction may comprise 1-10¹⁰ different catalytic molecules (each with a specific toehold domain sequence, for example). In some embodiments, a multiplexed primer exchange reaction comprises 1-10, 1-10², 1-10³, 1-10⁴, 1-10⁵, 1-10⁶, 1-10⁷, 1-10⁸, 1-10⁹, 1-10¹⁰, or more, different catalytic molecules. In some embodiments, a multiplexed primer exchange reaction comprises 1-5, 1-10, 1-15, 1-20, 1-25, 1-30, 1-35, 1-40, 1-45, 1-50, 1-55, 1-60, 1-65, 1-70, 1-75, 1-80, 1-85, 1-90, 1-95, 1-100, 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 10-15, 10-20, 10-25, 10-30, 10-35, 10-40, 10-45, 10-50, 10-55, 10-60, 10-65, 10-70, 10-75, 10-80, 10-85, 10-90, 10-95 or 10-100 different catalytic molecules. In some embodiments, a multiplexed primer exchange reaction comprises 1, 2, 3, 4, 5, 6, 7, 8, 9, 10, 11, 12, 13, 14, 15, 16, 17, 28, 19, 20, 21, 22, 23, 24 or 25 different catalytic molecules. Catalytic molecules are different from each other if their toehold domains differ from each other, for example.

The kinetics of a multiplexed primer exchange reaction may be controlled by varying temperature, time, buffer/salt conditions, and deoxyribonucleotide triphosphate (dNTP) concentrations, for example. Polymerases, like most enzymes, are sensitive to many buffer conditions, including ionic strength, pH and types of metal ions present (e.g., sodium ions vs. magnesium ions). Thus, the temperature at which a multiplexed primer exchange reaction is performed may vary from, for example, 4° C. to 65° C. (e.g., 4° C., 25° C., 37° C., 42° C. or 65° C.). In some embodiments, the temperature at which a multiplexed primer exchange reaction is performed is 4-25° C., 4-30° C., 4-35° C., 4-40° C., 4-45° C., 4-50° C., 4-55° C., 4-60° C., 10-25° C., 10-30° C., 10-35° C., 10-40° C., 10-45° C., 10-50° C., 10-55° C., 10-60° C., 25-30° C., 25-35° C., 25-40° C., 25-45° C., 25-50° C., 25-55° C., 25-60° C., 25-65° C., 35-40° C., 35-45° C., 35-50° C., 35-55° C., 35-60° C., or 35-65° C. In some embodiments, a multiplexed primer exchange reaction is performed at room temperature, while in other embodiments, a multiplexed primer exchange reaction is performed at 37° C.

In some embodiments, a primer exchange reaction may be performed (incubated) for 30 minutes (min) to 24 hours (hr). In some embodiments, a multiplexed primer exchange reaction is carried out for 10 min, 35 min, 40 min, 45 min, 50 min, 55 min, 60 min, 1 hr, 2 hr, 3 hr, 4 hr, 5 hr, 6 hr, 7 hr, 8 hr, 9 hr, 10 hr, 11 hr, 12 hr, 18 hr or 24 hr.

Deoxyribonucleotides (dNTPs) are the “fuel” that drives a multiplexed primer exchange reaction. Thus, the kinetics of a primer exchange reaction, in some embodiments, depends heavily on the concentration of dNTPs in a reaction. The concentration of dNTPs in a multiplexed primer exchange reaction may be, for example, 2-1000 μM. In some embodiments, the dNTP concentration in a multiplexed primer exchange reaction is 2-10 μM, 2-15 μM, 2-20 μM, 2-25 μM, 2-30 μM, 2-35 μM, 2-40 μM, 2-45 μM, 2-50 μM, 2-55 μM, 2-60 μM, 2-65 μM, 2-70 μM, 2-75 μM, 2-80 μM, 2-85 μM, 2-90 μM, 2-95 μM, 2-100 μM, 2-110 μM, 2-120 μM, 2-130 μM, 2-140 μM, 2-150 μM, 2-160 μM, 2-170 μM, 2-180 μM, 2-190 μM, 2-200 μM, 2-250 μM, 2-300 μM, 2-350 μM, 2-400 μM, 2-450 μM, 2-500 μM, 2-600 μM, 2-700 μM, 2-800 μM, 2-900 μM or 2-1000 μM. For example, the dNTP concentration in a multiplexed primer exchange reaction may be 2 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 105 μM, 110 μM, 115 μM, 120 μM, 125 μM, 130 μM, 135 μM, 140 μM, 145 μM, 150 μM, 155 μM, 160 μM, 165 μM, 170 μM, 175 μM, 180 μM, 185 μM, 190 μM, 195 μM or 200 μM. In some embodiments, the dNTP concentration in a primer exchange reaction is 10-20 μM, 10-30 μM, 10-40 μM, 10-50 μM, 10-60 μM, 10-70 μM, 10-80 μM, 10-90 μM or 10-100 μM.

In some embodiments, dNTP variants are used. For example, PER systems may use hot start/clean amp dNTPs, phosphorothioate dNTPs, or fluorescent dNTPs. Other dNTP variants may be used. Because some modified dNTPs are less favorable than normal (unmodified) DNA-DNA binding, the hairpin back displacement process may be increased with their usage. Similarly, a hairpin comprised of a different type of nucleic acid (e.g., LNA, RNA or interspersed modified bases such as methyl dC or super T IDT modifications) may be used in some embodiments to increase the speed of a PER by forming stronger bonds than the synthesized primer with respect to the catalytic molecule.

In some embodiments, catalytic molecules are covalently linked to biomolecules such as fluorophores or proteins. In some embodiments, catalytic molecules contain a biotin modification, so they may be tethered to surfaces by a biotin-streptavidin bond. In some embodiments, catalytic molecules contain a modification such as an azide modification within one of the subdomains that allows them to be covalently linked to other molecules or to the matrix, such as linked to an alkyne through click chemistry. Other chemical and biological linkages are encompassed by the present disclosure.

(iii) Multiplex PER

In some aspects, the methods provided herein comprises contacting the sample with a plurality of initial hairpin molecules for targeting a plurality of analytes, wherein each analyte of the plurality of analytes is or is associated with a nucleic acid molecule (PER primer) comprising a free 3′ priming region, wherein each initial hairpin molecule of the plurality comprises (1) an unpaired 3′ toehold domain, (2) a paired stem domain formed by intramolecular nucleotide base pairing between a 3′ paired stem subdomain of the initial hairpin molecule and a 5′ paired stem subdomain of the initial hairpin molecule, and (3) a hairpin loop domain, and wherein the 3′ toehold domain is complementary to the free 3′ priming region of an analyte or associated with an analyte of the plurality of analytes. In some embodiments, each of the PER primers is directly or indirectly immobilized to the matrix, such that the position of each PER primer is preserved in the matrix (and thus, the position of the corresponding analyte can be identified). In some embodiments, a set of corresponding nucleic acid molecule (PER primer) comprising a free 3′ priming region (optionally associated with a labelling and capture agent) and hairpin molecules for each analyte of interest is used.

In some embodiments, the method comprises providing conditions for polymerization to produce elongated products of the plurality of immobilized nucleic acid molecules, wherein the plurality of immobilized nucleic acid molecules correspond to the plurality of analytes. In some embodiments, the free 3′ priming region of each nucleic acid molecule uniquely identifies the target analyte (e.g., the nucleic acid molecules are orthogonal primers). In some embodiments, the plurality of initial hairpin molecules each comprise an unpaired 3′ toehold domain sequence that uniquely corresponds to a free 3′ priming region of a nucleic acid molecule associated with an analyte in the sample. For example, the initial hairpin molecules can be designed to avoid cross-hybridization with 3′ ends of other nucleic acid molecules in the sample. Methods for designing orthogonal PER catalytic molecules (e.g., hairpin molecules) for multiplexed PER reactions have been described, for example, in Saka, S. K., et al. (2019). “Immuno-SABER enables highly multiplexed and amplified protein imaging in tissues”. Nat Biotechnol 37, 1080-1090; Kishi, J. Y. et al. (2019). “SABER amplifies FISH: enhanced multiplexed imaging of RNA and DNA in cells and tissues”. Nature methods, 16(6), 533-544; U.S. Pat. Pub. 20190106733; and U.S. Pat. Pub 20200362398, the entire contents of each of which are incorporated herein by reference.

In some embodiments, a nucleic acid molecule (PER primer) of a plurality of PER primers is associated with an analyte of the plurality of analytes (e.g., is hybridized to a sequence of the analyte), and the free 3′ priming region of the nucleic acid molecule comprises a sequence (e.g., a barcode sequence) that corresponds to the analyte. In some embodiments, an initial hairpin molecule of the plurality of initial hairpin molecules comprises a sequence that corresponds to an analyte of the plurality of analytes. In some embodiments, the unpaired 3′ toehold domain of an initial hairpin molecule of the plurality comprises a sequence that uniquely corresponds to an analyte of the plurality of analytes. In some embodiments, the 3′ subdomain of a hairpin molecule paired stem comprises a sequence that corresponds to an analyte of the plurality of analytes. In some embodiments, an initial hairpin molecule of the plurality comprises a barcode sequence that corresponds to an analyte of the plurality of analytes. In some embodiments, the elongated products produced from the plurality of immobilized nucleic acid molecules each comprise a repeating nucleotide sequence domain (e.g., a barcode sequence) corresponding to an analyte of the plurality of analytes. In some embodiments, the analytes are identified by combinatorial or sequential hybridization of detection probes to the elongated products. In any of the preceding embodiments, the sequence corresponding to the analyte of the plurality of analytes can uniquely correspond to the analyte of the plurality of analytes.

In some embodiments, the method comprises contacting the sample with a capture agent that interacts with an analyte of the plurality of analytes in the sample and immobilizing the capture agent to the matrix. In some embodiments, the capture agent interacts with multiple analytes of the plurality of analytes. For example, the capture agent can comprise an oligo dT or poly T sequence, wherein the capture agent hybridizes to nucleic acid analytes comprising a poly A sequence, thereby immobilizing polyadenylated RNA molecules in the matrix.

### (iv) Detection

According to one aspect, the three dimensional matrix material is optically transparent. According to one aspect, the three dimensional matrix material is optically transparent to allow for three dimensional imaging techniques. In some aspects, the methods provided herein allow for detection of an amplified signal associated with an analyte, wherein an elongated product of a primer exchange reaction immobilized in the matrix provides a scaffold for hybridization of multiple detection probes comprising detectable moieties and/or one or more secondary probes (e.g., secondary PER primers or concatemers).

In some embodiments, a barcode sequence in a hairpin molecule that corresponds to an analyte of the plurality of analytes can be detected. In some embodiments, the elongated products produced from the plurality of immobilized nucleic acid molecules each comprise a repeating nucleotide sequence domain (e.g., a barcode sequence) corresponding to an analyte of the plurality of analytes can be detected. In some embodiments, the analytes are identified by combinatorial or sequential hybridization of detection probes to the elongated products. In any of the preceding embodiments, the sequence corresponding to the analyte of the plurality of analytes can uniquely correspond to the analyte of the plurality of analytes. For example, the one or more barcode sequences can be detected by hybridizing a corresponding intermediate probe (e.g., L-shaped or U-shaped probe) comprising an overhang region that can bind to a detectably-labelled probe (e.g., detection probe). In any of the preceding embodiments, the detecting can comprise contacting the sample with a detection probe, wherein the detection probe comprises a detectable moiety; wherein the detection probe hybridizes directly or indirectly (e.g., via an intermediate probe) to the elongated product (e.g., a barcode sequence associated with the analyte) and detecting the detectable moiety of the detection probe, thereby detecting the elongated product. In some embodiments, a plurality of the detection probes can hybridize sequentially to the elongated product and be detected sequentially.

In some embodiments, the method comprises assigning a signal code sequence to the barcode sequence in the PER concatemer, and sequentially contacting the biological sample with probes comprising recognition sequences complementary to a sequence of the barcode sequence, wherein each of the probes is associated with a signal or absence thereof that provides a “signal code” as part of the signal code sequence, until sufficient signals have been detected to determine the signal code sequence. In some embodiments, the probes comprising recognition sequences comprise one or more overhang regions that do not hybridize to the hairpin molecule (e.g., an overhang region at one end of the probe, as in an L-shaped probe, or an overhang region at both ends of the probe, as in a U-shaped probe). In some embodiments, the overhang region comprises one or more binding sites for a detection probe. Thus, the probes comprising recognition sequences can be associated with signals by hybridization of a detection probe to the overhang region of the probes comprising recognition sequences. In some embodiments, the detecting step comprises multiplex detection of PER concatemers in the matrix, wherein the method comprises sequential hybridization steps to determine signal code sequences assigned to a plurality of different barcode sequences associated with the plurality of PER products. In some embodiments, a sequential hybridization step comprises contacting the matrix with a pool of probes comprising different recognition sequence corresponding to the different barcode sequences, and a pool of detection probes comprising different detectable moieties. In some embodiments, the pool of detection probes is smaller than the pool of probes comprising different recognition sequences (e.g., the pool of detection probes comprises no more than 4, no more than 5, or no more than 6 different detection probes corresponding to different detectable moieties, and the pool of probes comprises at least 10, at least 20, at least 50, or at least 100 different recognition sequences). In some embodiments, subsequent sequential hybridization steps comprise contacting the matrix with a different pool of detection probes corresponding to subsequent signal codes of the signal code sequence, but with the same pool of detection probes.

One or more dark cycles can be incorporated into decoding the barcode sequences by contacting the matrix with a probe that is not detectably labeled and/or does not comprise a binding site for a detection probe. In some aspects, the absence of a detectable signal may be used as a “color,” e.g., in addition to the limited number of available fluorescent color channels in fluorescent microscopy, dark cycles can be used to alleviate issues associated with optical crowding in one or more color channels. In some embodiments, the signal code sequence includes one or more dark cycles as a “signal code” in the signal code sequence. Different probes may be detected, or distinguished from one another, by different labels, or by absence of a detectable label. In some embodiments, a probe may be directly or indirectly labelled with a detectable label which gives rise to a signal which may be recorded and/or assigned (e.g., serially) a signal code. In some embodiments, a probe is capable of hybridizing to a different target nucleic acid sequence (e.g., barcode sequence corresponding to a target analyte) and providing a signal. In some embodiments, a signal may include the signal detectable from the detectable label, and different detectable labels may provide different signals which may be distinguished, e.g. by color. In some embodiments, absence of signal may also be recorded and/or assigned a signal code. In some embodiments, in a plurality of probes, one or more of the probes may be lacking a detectable label, and thus the absence of a signal may be recorded and analyzed, for example, by assigning a signal code to the absence of signal (also known as a “dark” cycle for the one or more of the probes and the corresponding analyte(s)). In some embodiments, when there is a single cycle of detection to detect the signals from the probes, the plurality of probes may comprise molecules of one probe which is associated with the absence of a signal, and the remainder of the probes may be associated with detectable labels which can be distinguished from one another (e.g., the probes may be detectably labeled with distinguishable labels, or may comprise distinct sequences for hybridizing to detectably labeled probes comprising distinguishable labels). In some embodiments, a combinatorial, e.g. sequential, labelling scheme is used (e.g., multiple cycles of sequential signal detection), and the plurality of probes for different barcode sequences used in a given cycle need not all be distinguishable from one another in terms of the signal (e.g., may comprise the same detectable label, such as the same color of fluorophore), as it is the combination (e.g., sequence or order) of signals which identifies the barcode sequence, not a single signal.

In some embodiments, a detecting step of the method can comprise: (i) contacting the sample with a detection probe, wherein the detection probe comprises a detectable moiety; wherein the detection probe hybridizes directly or indirectly to the elongated product; and (ii) detecting the detectable moiety of the detection probe, thereby detecting the elongated product. In some embodiments, the detection probe can hybridize to the repeating nucleotide sequence domain. In some embodiments, the detection probe can hybridize indirectly to the elongated product, optionally wherein the detection probe hybridizes to a repeating sequence of a secondary PER concatemer that is hybridized to the elongated product. In some embodiments, the secondary PER concatemer can be formed in situ in the hydrogel matrix according to the methods described herein. In some embodiments, the secondary PER concatemer can be formed in vitro and added to the matrix pre-formed. In some embodiments, the detectable moiety of the signal strands is a fluorophore. In some embodiments, each of the signal strands has a length of 10-30 nucleotides (e.g., 10-15 nucleotides, 15-20 nucleotides, 20-25 nucleotides, or 25-30 nucleotides).

PER strands can be applied in a cascade fashion to form branched structures through the hybridization of additional concatemers onto the primary concatemer. Formation of the branches can be performed by simultaneous or sequential application of the pre-concatemers to the target. Branched concatemers may also be formed by direct in situ PER. The creation of branches increases the number of binding sites for the fluorescent oligonucleotides, enabling further signal amplification.

In some embodiments, the analysis and/or sequence determination involves washing to remove unbound polynucleotides (e.g., primary, secondary, or tertiary detectably-labelled probes), thereafter revealing a fluorescent product for imaging.

In some embodiments, the detection comprises imaging the sample or a region thereof in each of the detection steps. In some aspects, the imaging can be performed using any suitable means and systems. In some examples, the imaging is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectably-labelled probes. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

The spatial movement limit of an analyte (or a product or derivative thereof) in a sample allowed for a temporal detection of the detectably-labelled probes to occur can vary depending on a number of factors, including, but not limited to, presence of any distinguishable features within a field of detection, magnification used in detection (e.g., magnification of the microscope lens), density of the analytes in a sample, and any combinations thereof. In some embodiments, there can be no limit in the spatial movement of an analyte in a sample during a temporal detection of the detection reagents, for example, provided that the analyte stay within the field of detection and there is at least one same distinguishable feature in each image taken during a temporal detection so that the images can be aligned to each other based on the same distinguishable feature. In some embodiments where there is no such distinguishable feature, the spatial movement of an analyte in a sample can be less than 100 including less than 50 less than 25 less than 10 less than 1 μm or smaller, over a time period, during which a temporal detection of the detection reagents occurs. In some embodiments, the spatial movement of an analyte in a sample can be less than 1000 nm, including less than 500 nm, less than 250 nm, less than 100 nm, less than 50 nm, less than 10 nm or smaller, over a time period, during which a temporal detection of the labelling agents occurs. More importantly, the spatial movement limit of an analyte in a sample during a temporal detection is determined by the ability of matching distinguishable features between images taken during a temporal detection, which can be affected by imaging conditions. In some embodiments, the analyte can be fixed on a solid substrate or support.

In some aspects, an assay may comprise a series of probe hybridizations and probe detection, thereby generating a sequential code of detectable signals that can be used to identify the target analyte. Suitable methods for sequential hybridization and detection are described in Section II C above.

### IV. Compositions and Kits

In some aspects, provided herein are hybridization complexes comprising any of the nucleic acid molecules and hairpin primers disclosed herein, wherein the nucleic acid molecule comprises a functional moiety for immobilization to a matrix or is associated with an agent (e.g., capture or labelling agent) that comprises a functional moiety for immobilization to a matrix. In some embodiments, an analyte in the sample can be immobilized to a matrix.

Also provided herein are kits, for analyzing an analyte in a biological sample embedded in a three-dimensional polymerized matrix according to any of the methods described herein. In some embodiments, provided herein is a kit comprising: a) a nucleic acid molecule, wherein the nucleic acid molecule comprises a free 3′ priming region, optionally wherein the nucleic acid molecule is associated with a labelling agent that binds to an analyte in the sample or is a probe that binds to the analyte in the sample; b) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the nucleic acid molecule is complementary to the 3′ toehold domain of the initial hairpin molecule; and c) a matrix-forming material. In some embodiments, the nucleic acid molecule or capture agent or labelling agent comprises a functional moiety for immobilization to the matrix, optionally wherein the functional moiety is selected from the group consisting of an amine, acrydite, alkyne, biotin, azide, or thiol.

In some aspects, provided herein is a kit comprising a nucleic acid molecule, wherein the nucleic acid molecule comprises a free 3′ priming region; b) a capture agent that interacts with an analyte in the sample, optionally wherein the nucleic acid molecule is associated with a labelling agent that binds to the analyte or capture agent in the sample or is a probe that binds to the analyte or capture agent in the sample; c) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain, wherein the free 3′ priming region of the nucleic acid molecule is complementary to the 3′ toehold domain of the initial hairpin molecule; and d) a matrix-forming material. In some embodiments, the capture agent comprises a functional moiety for immobilization to the matrix, optionally wherein the functional moiety is selected from the group consisting of an amine, acrydite, alkyne, biotin, azide, or thiol.

In some aspects, the kit further comprises one or more reagents for performing a primer exchange reaction, such as a polymerase having strand-displacing activity and/or dNTPs. In some embodiments, the kit further comprises detection probes designed to hybridize to a repeating sequence of an elongated product generated using the nucleic acid molecule and hairpin molecule in a primer exchange reaction. In some embodiments, the kit comprises a set of reagents for forming and detecting an elongated product corresponding to each analyte.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### V. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

A “primer extension” refers to any method where two nucleic acid sequences (e.g., a constant region from each of two distinct capture probes) become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™ ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™ ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)2 fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “detectable moiety”, “optical label,” and “label” are used interchangeably herein to refer to a directly or indirectly detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, capture probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7- Amino-4-methylcoumarin, 6-Aminoquinoline, Aniline Blue, ANS, APC-Cy7, ATTO-TAG™ CBQCA, ATTO-TAG™ FQ, Auramine 0-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (Di1C18(5)), DIDS, Dil (DilC18(3)), DiO (DiOC18(3)), DiR (Di1C18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOY041)-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and- methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### Example

The following example is included for illustrative purposes only and is not intended to limit the scope of the present disclosure.

### Example 1: Design and Elongation of PER primers

This example describes various designs of nucleic acid molecules for use in a primer exchange reaction, wherein the PER primers (depicted in FIGS. 1-4 as “nucleic acid molecule”) tested comprises a free 3′ priming end. Various primer lengths, spacers, and repeats were tested in the PER primer design.

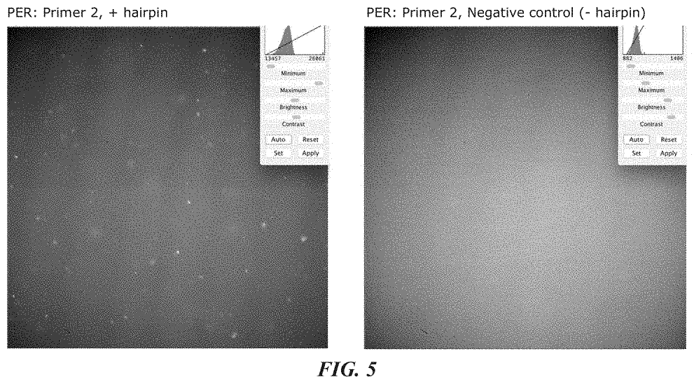

To test PER primer elongation when immobilized to a matrix, gel casting was performed with nucleic acid molecules of various lengths and sequences (e.g., Primers 1˜4 provided as SEQ ID Nos: 1-4 in Table 1) with a 5′ acrydite moiety for immobilization to the matrix. The gel was cooled and a PER reaction mixture was prepared (PBS, MgSO₄, dNTPs, Bst DNA Polymerase, and hairpin molecules). The PER reaction mixture was added to the gel and incubated with the gel for elongation. Elongated products were detected by adding an imager nucleic acid molecule (SEQ ID NO:5 and 6 in Table 1). In the presence of the hairpin molecules compared to negative control samples where hairpin molecules were not provided, primers 2-4 (SEQ ID Nos: 2-4) were observed to generate elongation products in the matrix. For example, FIG. 5 shows elongation products generated in the matrix using Primer 2 in the left panel where hairpin molecules were provided.

TABLE 1
Nucleic acid sequences for PER primer design

| | SEQ | |
|---|---|---|
| Oligo | ID NO | Sequence |
| Primer 1 | 1 | /5′ Acrydite/ttttttttttCCAATAATA |
| Primer 2 | 2 | /5′Acrydite/ttttttttttACCAATAATA |
| | | ACCAATAATA ACCAATAATA |
| Primer 3 | 3 | /5′ Acrydite/ttttttttttCATCATCAT |
| Primer 4 | 4 | /5′Acrydite/ttttttttttA CATCATCAT |
| | | ACATCATCAT ACATCATCAT |
| Imager | 5 | /5′ Cy3 dye/tt TATTATTGGT |
| molecule 1 | | TATTATTGGT /3′ Inverted dT/ |
| Imager | 6 | /5′ Cy3 dye /tt ATGATGATGT |
| molecule 2 | | ATGATGATGT /3′ Inverted dT/ |

In an experiment to test various primers (depicted schematically in FIGS. 1-4 as “nucleic acid molecule”) with primer and hairpin sequences as set forth in Table 2, it was observed that PER elongation products were formed only in the presence of the primer but not in a negative control where primer molecules were not provided. In conclusion from the experiments provided, it was observed that both primer and hairpin molecules are necessary for PER elongation. Various sequences were tested for the primer sequence for elongation. It was observed that no PER product was generated in the absence of the primer.

TABLE 2
Nucleic acid sequences for PER

| | SEQ | |
|---|---|---|
| Oligo | ID NO | Sequence |
| Primer 5 | 7 | CCAATAATA |
| Primer 6 | 8 | CGTAACCAAGCGTAGTATCGACCAATAATA |
| Hairpin | 9 | ACCAATAATAGGGCCTTTTGGCCCTATTATTG |
| | | GTTATTATTGG/3′ Inverted dT/ |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method for analyzing a sample, comprising:
a) contacting the sample with a matrix-forming material to embed the sample in a three-dimensional polymerized matrix, wherein a nucleic acid molecule in the sample comprises a free 3′ priming region;
b) immobilizing the nucleic acid molecule to the matrix;
c) contacting the sample with:
(i) an initial hairpin molecule comprising (1) an unpaired 3′ toehold domain, (2) a paired stem domain formed by intramolecular nucleotide base pairing between a 3′ subdomain of the initial hairpin molecule and a 5′ subdomain of the initial hairpin molecule, and (3) a hairpin loop domain,
wherein the free 3′ priming region of the nucleic acid molecule is complementary to the 3′ toehold domain of the initial hairpin molecule, and
(ii) a polymerase having strand displacement activity;
d) incubating the sample under conditions for polymerization to produce an elongated product of the immobilized nucleic acid molecule; and
e) detecting the elongated product that is immobilized to the matrix.

2. The method of claim 1, wherein the sample is contacted with
a capture agent that interacts with an analyte in the sample, and immobilizing the capture agent or the analyte is immobilized to the matrix.

3. (canceled)

4. The method of claim 2, wherein the nucleic acid molecule is associated with a labelling agent that binds the analyte and/or the capture agent.

5. (canceled)

6. The method of claim 2, wherein the nucleic acid molecule is a probe that hybridizes to the analyte or to a target nucleic acid sequence associated with the analyte.

7.-8. (canceled)

9. The method of claim 6, wherein the nucleic acid molecule comprises a target hybridization region that hybridizes to the analyte or to the target nucleic acid sequence associated with the analyte.

10. The method of claim 9, wherein the nucleic acid molecule is immobilized to the matrix at an attachment point, and wherein the target hybridization region is located between the attachment point and the free 3′ priming region.

11. The method of claim 9, wherein the nucleic acid molecule is immobilized to the matrix at an attachment point, and wherein the target hybridization region is located 5′ of the attachment point and the free 3′ priming region.

12. (canceled)

13. The method of claim 2,
wherein the nucleic acid molecule is an endogenous nucleic acid molecule or a product thereof, and wherein the endogenous nucleic acid molecule interacts with the capture agent.

14.-15. (canceled)

16. The method of claim 13, wherein the endogenous nucleic acid molecule is a messenger RNA (mRNA) molecule, wherein the capture agent comprises an oligo dT priming sequence, and wherein the immobilizing step further comprises generating a complementary DNA (cDNA) product using the mRNA as a template.

17. The method of claim 16, wherein the nucleic acid molecule is the cDNA product, and the initial hairpin molecule is designed to hybridize to the 3′ end of the cDNA product.

18. The method of claim 1, wherein the unpaired 3′ toehold domain of the initial hairpin molecule is complementary to a 5′ paired stem subdomain of the initial hairpin molecule.

19. The method of claim 1, further comprising contacting the sample with
a second hairpin molecule comprising: (i) an unpaired 3′ toehold domain, (ii) a paired stem domain formed by intramolecular nucleotide base pairing between a 3′ paired stem subdomain of the second hairpin molecule and a 5′ paired stem subdomain of the second hairpin molecule, and (iii) a loop domain, wherein the 3′ toehold domain of the second hairpin molecule is complementary to the 5′ subdomain of the initial hairpin molecule.

20. (canceled)

21. The method of claim 1, wherein the elongated product produced in (d) comprises a repeating nucleotide sequence domain, wherein the repeating domain is complementary to the stem domain of the initial and/or second hairpin molecule.

22. The method of claim 2, wherein the nucleic acid molecule or capture agent is immobilized to the matrix via a functional moiety that can be covalently cross-linked, copolymerized with, or otherwise non-covalently bound to the matrix.

23. (canceled)

24. The method of claim 1, wherein the nucleic acid molecule is immobilized to the matrix at an attachment point, and the nucleic acid molecule comprises a spacer region between the attachment point and the free 3′ priming region.

25.-29. (canceled)

30. The method of claim 1, wherein the detecting comprises:
(i) contacting the sample with a detection probe, wherein the detection probe comprises a detectable moiety; wherein the detection probe hybridizes directly or indirectly to the elongated product; and
(ii) detecting the detectable moiety of the detection probe, thereby detecting the elongated product.

31. (canceled)

32. The method of claim 1, wherein the method comprises contacting the sample with a matrix-forming material and using the matrix-forming material to form the matrix, and wherein the sample is contacted with the matrix-forming material prior to or concurrently with contacting the sample with the nucleic acid molecule or capture agent.

33.-35. (canceled)

36. The method of claim 1, wherein the matrix is functionalized with a cross-linker reactive group selected from the group consisting of acrydite, NHS ester, azide, maleimide, amine, and carboxyl groups.

37.-38. (canceled)

39. The method of claim 1, wherein the method comprises:
a) contacting the sample with a plurality of initial hairpin molecules for targeting a plurality of analytes,
wherein each analyte of the plurality of analytes is or is associated with a nucleic acid molecule comprising a free 3′ priming region,
wherein each initial hairpin molecule of the plurality comprises (1) an unpaired 3′ toehold domain, (2) a paired stem domain formed by intramolecular nucleotide base pairing between a 3′ paired stem subdomain of the initial hairpin molecule and a 5′ paired stem subdomain of the initial hairpin molecule, and (3) a hairpin loop domain, and wherein the 3′ toehold domain is complementary to the free 3′ priming region of an analyte or associated with an analyte of the plurality of analytes; and
b) providing conditions for polymerization to produce elongated products of a plurality of immobilized nucleic acid molecules, wherein the plurality of immobilized nucleic acid molecules correspond to the plurality of analytes.

40.-46. (canceled)

47. The method of claim 1, wherein the biological sample is a tissue sample.

48.-56. (canceled)


SEQUENCE LISTING (9 sequences)

SEQ ID NO: 1 - DNA, 19 bp, Artificial Sequence
  Synthetic Construct
ttttttttttccaataata

SEQ ID NO: 2 - DNA, 40 bp, Artificial Sequence
  Synthetic Construct
ttttttttttaccaataataaccaataataaccaataata

SEQ ID NO: 3 - DNA, 19 bp, Artificial Sequence
  Synthetic Construct
ttttttttttcatcatcat

SEQ ID NO: 4 - DNA, 40 bp, Artificial Sequence
  Synthetic Construct
ttttttttttacatcatcatacatcatcatacatcatcat

SEQ ID NO: 5 - DNA, 23 bp, Artificial Sequence
  modified_base 23: Final T is inverted
  Synthetic Construct
tttattattggttattattggtt

SEQ ID NO: 6 - DNA, 23 bp, Artificial Sequence
  modified_base 23: Final T is inverted
  Synthetic Construct
ttatgatgatgtatgatgatgtt

SEQ ID NO: 7 - DNA, 9 bp, Artificial Sequence
  Synthetic Construct
ccaataata

SEQ ID NO: 8 - DNA, 30 bp, Artificial Sequence
  Synthetic Construct
cgtaaccaagcgtagtatcgaccaataata

SEQ ID NO: 9 - DNA, 44 bp, Artificial Sequence
  modified_base 44: Final T is inverted
  Synthetic Construct
accaataatagggccttttggccctattattggttattattggt